(19) United States
(12) Patent Application Publication
(10) Pub. No.: US 2023/0404514 A1
(43) Pub. Date: Dec. 21, 2023

(54) MEDICAL DATA PROCESSING METHOD, MODEL GENERATING METHOD, AND MEDICAL DATA PROCESSING APPARATUS

(71) Applicant: CANON MEDICAL SYSTEMS CORPORATION, Otawara-shi (JP)

(72) Inventors: Masakazu MATSUURA, Nasushiobara (JP); Takuya NEMOTO, Hitachinaka (JP); Hiroki TAGUCHI, Otawara (JP); Yuto HAMADA, Nasushiobara (JP); Yohei MINATOYA, Bunkyo (JP)

(73) Assignee: CANON MEDICAL SYSTEMS CORPORATION, Otawara-shi (JP)

(21) Appl. No.: 18/332,006

(22) Filed: Jun. 9, 2023

(30) Foreign Application Priority Data
Jun. 16, 2022 (JP) 2022-097241
Mar. 23, 2023 (JP) 2023-047004

(51) Int. Cl. A61B 6/00; G16H 30/40; G06N 3/0464; G06N 3/08

(52) U.S. Cl. CPC A61B 6/5258; A61B 6/405; A61B 6/482; A61B 6/5205; G16H 30/40; G06N 3/0464; G06N 3/08

(57) ABSTRACT

A medical data processing method according to an embodiment includes: outputting second spectral data by inputting first spectral data related to an examined subject imaged by a spectral medical imaging apparatus to a trained model configured to generate, on the basis of the first spectral data, the second spectral data having less noise than the first spectral data and a higher resolution than the first spectral data. The first spectral data in the medical data processing method according to the embodiment corresponds to medical data obtained by performing a spectral scan on the examined subject. The trained model in the medical data processing method according to the embodiment is configured to perform a noise reducing process and a super-resolution process on the first spectral data.

### CROSS-REFERENCE TO RELATED APPLICATIONS

This application is based upon and claims the benefit of priority from Japanese Patent Application No. 2022-097241, filed on Jun. 16, 2022; and Japanese Patent Application No. 2023-47004, filed on Mar. 23, 2023, the entire contents of which are incorporated herein by reference.

### FIELD

Embodiments described herein relate generally to a medical data processing method, a model generating method, and medical data processing apparatus.

### BACKGROUND

Conventionally, in Computed Tomography (CT) medical examinations using an X-ray Computed Tomography (CT) apparatus, for example, lung fields and bones require observation on minute structures. For this reason, in CT examinations of lung fields and bones, CT images reconstructed by an X-ray CT apparatus are required to have higher spatial resolutions than images of other sites. Further, for example, Dual Energy (DE) CT apparatuses and Photon Counting (PC) CT apparatuses are configured to obtain energy information of X-rays. For this configuration, DECT apparatuses and PCCT apparatuses are equipped with spectral imaging technology for performing an image reconstruction with substance discrimination. For image processing related to the spectral imaging, for example, a Filtered Backprojection (FBP)-based reconstruction method is known to use a technique by which the spatial resolution of a reconstructed CT image is enhanced by using a reconstruction mathematical function that strengthens radio frequencies. Further, in recent years, a super-resolution technique is proposed by which a trained model using deep learning is aimed to enhance spatial resolutions in spectral imaging.

However, in spectral imaging, according to the FBP-based spatial resolution enhancing technique using the reconstruction mathematical function, a radio frequency component is emphasized throughout the entire reconstructed image. For this reason, according to the FBP-based spatial resolution enhancing technique, noise may be emphasized at the same time, which may worsen visibility of anatomical structures in the reconstructed CT image. In contrast, in a super-resolution (higher resolution) CT image obtained by a trained model using deep learning, because it is possible to selectively enhance resolutions of anatomical structures, it is possible to solve the problem of the FBP-based reconstruction method.

Further, in spectral imaging, when a DECT apparatus has acquired projection data by using a lower radiation dose, the projection data has more noise than projection data acquired with a higher radiation dose. For this reason, even when the resolution is enhanced with the super-resolution technique, the noise in a super-resolution CT image may worsen visibility of anatomical structures.

### BRIEF DESCRIPTION OF THE DRAWINGS

FIG. 1 is a diagram illustrating an exemplary configuration of a PCCT apparatus according to an embodiment;

FIG. 2 is a flowchart illustrating an example of a procedure in a noise-reduction super-resolution process according to the embodiment;

FIG. 3 is a diagram according to the embodiment illustrating an outline of the noise-reduction super-resolution process using first spectral data;

FIG. 4 is a diagram according to the embodiment illustrating an outline of a noise-reduction super-resolution process using projection data as an example of the first spectral data;

FIG. 5 is a diagram according to the embodiment illustrating an outline of a noise-reduction super-resolution process using a reconstructed image as an example of the first spectral data;

FIG. 6 is a diagram according to the embodiment illustrating an example in which a noise-reduction super-resolution process is applied to a first reconstructed image generated by a dual energy CT apparatus;

FIG. 7 is a diagram according to the embodiment illustrating an exemplary configuration of a training apparatus related to generating a noise-reduction super-resolution model;

FIG. 8 is a flowchart according to the embodiment illustrating an exemplary procedure in a process to generate the noise-reduction super-resolution model by training a Deep Convolution Neural Network (DCNN) while using first training data and second training data;

FIG. 9 is a diagram according to the embodiment illustrating an outline of a model generating process;

FIG. 10 is a table according to the embodiment illustrating examples of combinations of data subject to a noise simulation and a resolution simulation;

FIG. 11 is a diagram related to FIG. 10 (a) of the embodiment illustrating an outline of a model generating process in the situation where projection data is an input/output of a noise-reduction super-resolution model serving as a trained model;

FIG. 12 is a diagram related to FIG. 10 (a) of the embodiment illustrating an outline of a model generating process in the situation where image data (reconstructed images) is an input/output of a noise-reduction super-resolution model serving as a trained model;

FIG. 13 is a diagram illustrating an outline of the model generating process in FIG. 10 (b) of the embodiment;

FIG. 14 is a diagram illustrating an outline of the model generating process in FIG. 10 (c) of the embodiment;

FIG. 15 is a diagram illustrating an outline of the model generating process in FIG. 10 (d) of the embodiment;

FIG. 16 is a diagram according to a fourth application example of the embodiment illustrating an example in which a first higher energy monochrome image and a first lower energy monochrome image are generated from count projection data obtained by a PCCT apparatus; and

FIG. 17 is a diagram according to the fourth application example of the embodiment illustrating an example of an outline of a model generating process.

### DETAILED DESCRIPTION

A medical data processing method according to an embodiment includes: outputting second spectral data by inputting first spectral data related to an examined subject imaged by a spectral medical imaging apparatus to a trained model configured to generate, on the basis of the first spectral data, the second spectral data having less noise than the first spectral data and a higher resolution than the first spectral data. The first spectral data in the medical data processing method according to the embodiment corresponds to medical data obtained by performing a spectral scan on the examined subject. The trained model in the medical data processing method according to the embodiment is configured to perform a noise reducing process and a super-resolution process on the first spectral data.

A medical data processing method, a model generating method, a medical data processing apparatus, and a medical data processing program will be explained below, with reference to the accompanying drawings. In the following embodiments, some of the elements that are referred to by using the same reference characters are assumed to perform the same operations, and duplicate explanations thereof will be omitted, as appropriate. Further, to explain specific examples, the medical data processing apparatus according to certain embodiments will be described as being installed in a spectral medical imaging apparatus, for instance. Alternatively, the medical data processing apparatus according to other embodiments may be realized by a server apparatus capable of realizing a medical data processing method, i.e., a server apparatus capable of executing a medical data processing program.

The medical data processing apparatus will be described as being installed in a Photon Counting X-ray Computed Tomography (hereinafter, “Photon Counting Computed Tomography (PCCT) apparatus”) serving as an example of the spectral medical imaging apparatus. The spectral medical imaging apparatus in which the present medical data processing apparatus is installed does not necessarily have to be a PCCT apparatus and may be a Dual Energy (DE) CT apparatus, for example. Alternatively, the imaging apparatus may be a combination apparatus including a nuclear medicine diagnosis apparatus for Positron Emission Tomography (PET), Single Photon Emission Computed Tomography (SPECT), or the like combined with a spectral medical imaging apparatus.

### Embodiments

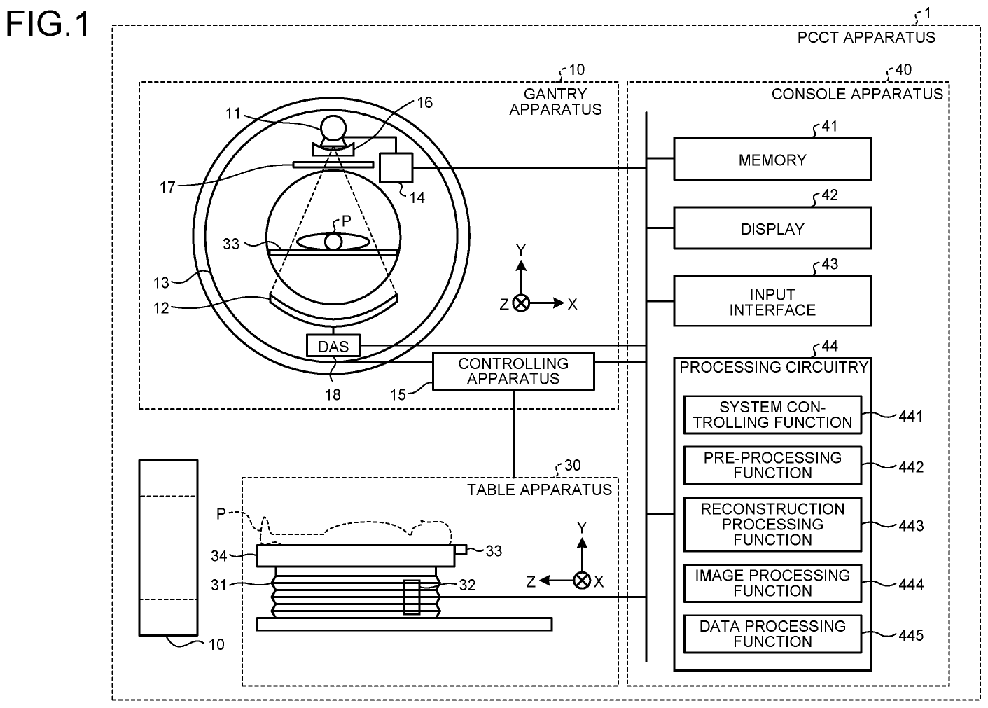

FIG. 1 is a diagram illustrating an exemplary configuration of a PCCT apparatus 1 according to an embodiment. As illustrated in FIG. 1, the PCCT apparatus 1 includes a gantry apparatus 10 which may be called a gantry, a table apparatus 30, and a console apparatus 40. The medical data processing apparatus according to the present embodiment corresponds to a configuration obtained by, for example, eliminating a system controlling function 441 and a pre-processing function 442 from the console apparatus 40 illustrated in FIG. 1. Further, the medical data processing apparatus according to the present embodiment may be a configuration obtained by eliminating unnecessary constituent elements, as appropriate, from the console apparatus 40 illustrated in FIG. 1. In the present embodiment, the longitudinal direction of a rotation axis of a rotating frame 13 in a non-tilt state is defined as a Z-axis direction; a direction being orthogonal to the Z-axis direction and extending from a rotation center to a pillar supporting the rotating frame 13 is defined as an X-axis; and a direction orthogonal to the Z-axis and to the X-axis is defined as a Y-axis. Although FIG. 1 illustrates the gantry apparatus 10 in multiple locations for the sake of convenience in the explanations, the PCCT apparatus 1 in actuality is structured to include the single gantry apparatus 10.

The gantry apparatus 10 and the table apparatus 30 are configured to operate on the basis of an operation from an operator received via the console apparatus 40 or an operation from the operator received via an operation unit provided for the gantry apparatus 10 or the table apparatus 30. The gantry apparatus 10, the table apparatus 30, and the console apparatus 40 are connected in a wired or wireless manner, so as to be able to communicate with one another.

The gantry apparatus 10 is an apparatus including an imaging system configured to radiate X-rays onto an examined subject (hereinafter, “patient”) P and to acquire projection data from detection data of X-rays that have passed through the patient P. The gantry apparatus 10 includes an X-ray tube 11, an X-ray detector 12, the rotating frame 13, an X-ray high-voltage apparatus 14, a controlling apparatus 15, a wedge 16, a collimator 17, and a Data Acquisition System (DAS) 18.

The X-ray tube 11 is a vacuum tube configured to generate X-rays by causing thermo electrons to be emitted from a negative pole (a filament) toward a positive pole (a target or an anode), with application of high voltage and a supply of a filament current from the X-ray high-voltage apparatus 14. As a result of the thermo electrons colliding with the target, the X-rays are generated. The X-rays generated at an X-ray tube focal point of the X-ray tube 11 go through an X-ray emission window of the X-ray tube 11 so as to be formed into a cone beam shape, for example, via the collimator 17 and emitted onto the patient P. For instance, examples of the X-ray tube 11 include a rotating anode X-ray tube configured to generate the X-rays by having the thermo electrons emitted onto a rotating anode.

The X-ray detector 12 is configured to detect photons in the X-rays generated by the X-ray tube 11. More specifically, the X-ray detector 12 is configured to detect, in units of the photons, the X-rays that were emitted from the X-ray tube 11 and have passed through the patient P and is configured to output an electrical signal corresponding to the amount of the X-rays to the DAS 18. For example, the X-ray detector 12 includes a plurality of columns of detecting elements in each of which a plurality of detecting elements (which may be called “X-ray detecting elements”) are arranged in a fan angle direction (which may be called a “channel direction”) along an arc while being centered on the focal point of the X-ray tube 11. In the X-ray detector 12, the plurality of columns of detecting elements are arranged flat, along the Z-axis direction. In other words, for example, the X-ray detector 12 has a structure in which the plurality of columns of detecting elements are arranged flat, along a cone angle direction (which may be called a row direction or a slice direction).

Examples of the PCCT apparatus 1 include various types such as: a Rotate/Rotate Type (a third-generation CT) in which the X-ray tube 11 and the X-ray detector 12 integrally rotate around the patient P; and a Stationary/Rotate Type (a fourth-generation CT) in which only the X-ray tube 11 rotates around the patient P, while a large number of X-ray detecting elements arrayed in a ring formation are fixed. It is possible to apply any type to the present embodiment.

The X-ray detector 12 is a direct-conversion type X-ray detector including a semiconductor element configured to convert incident X-rays into electrical charges. The X-ray detector 12 of the present embodiment includes, for example, at least one high-voltage electrode, at least one semiconductor crystal, and a plurality of read electrodes. The semiconductor element may be referred to as an X-ray converting element. The semiconductor crystal may be realized by using, for example, cadmium telluride (CdTe) or cadmium zinc telluride (“CZT”, CdZnTe), or the like. In the X-ray detector 12, electrodes are provided on two planes that are orthogonal to the Y direction and that oppose each other while the semiconductor crystal is interposed therebetween. In other words, in the X-ray detector 12, a plurality of anode electrodes (which may be called “read electrodes” or “pixel electrodes”) and a cathode electrode (which may be called “a common electrode”) are provided while the semiconductor crystal is interposed therebetween.

Between the read electrodes and the common electrode, bias voltage is applied. In the X-ray detector 12, when X-rays are absorbed by the semiconductor crystal, electron-hole pairs are formed. As a result of electrons moving to the positive pole side (i.e., the side of the anode electrodes (the read electrodes)), and the holes moving to the negative pole side (the side of the cathode electrode), a signal related to the X-ray detection is output from the X-ray detector 12 to the DAS 18.

Alternatively, the X-ray detector 12 may be an indirect-conversion type detector configured to indirectly convert the incident X-rays into electrical signals. The X-ray detector 12 is an example of an X-ray detecting unit.

The rotating frame 13 is an annular frame configured to support the X-ray tube 11 and the X-ray detector 12 so as to oppose each other and configured to rotate the X-ray tube 11 and the X-ray detector 12 via the controlling apparatus 15 (explained later). In addition to the X-ray tube 11 and the X-ray detector 12, the rotating frame 13 further includes and supports the X-ray high-voltage apparatus 14 and the DAS 18. The rotating frame 13 is rotatably supported by a non-rotating part (e.g., a fixed frame; not illustrated in FIG. 1) of the gantry apparatus 10. A rotating mechanism includes, for example, a motor configured to generate rotation driving power and a bearing configured to transmit the rotation driving power to the rotating frame 13 so as to cause the rotation. For example, the motor is provided in the non-rotating part. The bearing is physically connected to the rotating frame 13 and to the motor. Thus, the rotating frame 13 rotates in accordance with rotating power of the motor.

The rotating frame 13 and the non-rotating part are each provided with communication circuitry of a contactless or contact type, so that a unit supported by the rotating frame 13 is able to communicate with the non-rotating part and with apparatuses external to the gantry apparatus 10. For example, when optical communication is adopted as a contactless communication method, the detection data generated by the DAS 18 is transmitted, via optical communication, from a transmitter provided on the rotating frame 13 and including a Light Emitting Diode (LED), to a receiver provided in the non-rotating part of the gantry apparatus 10 and including a photodiode, so as to be further transferred by a transmitting mechanism from the non-rotating part to the console apparatus 40. Other examples of the communication method include contactless data transfer methods such as a capacity coupling method and a radio wave method, as well as a contact data transfer method using a slip ring and an electrode brush. The rotating frame 13 is an example of a rotating unit.

The X-ray high-voltage apparatus 14 includes: a high-voltage generating apparatus including electrical circuitry such as a transformer, a rectifier, and the like and having a function of generating the high voltage to be applied to the X-ray tube 11 and the filament current to be supplied to the X-ray tube 11; and an X-ray controlling apparatus configured to control output voltage corresponding to the X-rays to be emitted by the X-ray tube 11. The high-voltage generating apparatus may be of a transformer type or an inverter type. Further, the X-ray high-voltage apparatus 14 may be provided for the rotating frame 13 or may be provided so as to belong to the fixed frame of the gantry apparatus 10. The X-ray high-voltage apparatus 14 is an example of an X-ray high-voltage unit.

The controlling apparatus 15 includes processing circuitry having a Central Processing Unit (CPU) or the like and a driving mechanism such as a motor and an actuator or the like. As hardware resources thereof, the processing circuitry includes a processor such as the CPU or a Micro Processing Unit (MPU) and one or more memory elements such as a Read-Only Memory (ROM), a Random Access Memory (RAM), and/or the like. Alternatively, the controlling apparatus 15 may be realized by using a processor such as a Graphics Processing Unit (GPU), an Application Specific Integrated Circuit (ASIC), or a programmable logic device (e.g., a Simple Programmable Logic Device (SPLD), a Complex Programmable Logic Device (CPLD), or a Field Programmable Gate Array (FPGA)).

When the processor is a CPU, for example, the processor is configured to realize the functions by reading and executing programs saved in a memory. In contrast, when the processor is an ASIC, instead of having the programs saved in the memory, the functions are directly incorporated in the circuitry of the processor as logic circuitry. Further, the processors of the present embodiment do not each necessarily have to be structured as a single piece of circuitry. It is also acceptable to structure a single processor by combining together a plurality of pieces of independent circuitry, so as to realize the functions thereof. Furthermore, it is also acceptable to integrate two or more constituent elements into a single processor so as to realize the functions thereof.

The controlling apparatus 15 has a function of receiving input signals from an input interface attached to the console apparatus 40 or to the gantry apparatus 10 and controlling operations of the gantry apparatus 10 and the table apparatus 30. For example, upon receipt of the input signals, the controlling apparatus 15 is configured to exercise control to rotate the rotating frame 13, control to tilt the gantry apparatus 10, and control to bring the table apparatus 30 and a tabletop 33 into operation. In this situation, the control to tilt the gantry apparatus 10 may be realized as a result of the controlling apparatus 15 rotating the rotating frame 13 on an axis parallel to the X-axis direction, according to inclination angle (tilt angle) information input through an input interface attached to the gantry apparatus 10.

The controlling apparatus 15 may be provided for the gantry apparatus 10 or may be provided for the console apparatus 40. Further, instead of having the programs saved in the memory, the controlling apparatus 15 may be configured to directly incorporate the programs into the circuitry of a processor. The controlling apparatus 15 is an example of a controlling unit.

The wedge 16 is a filter for adjusting the X-ray amount of the X-rays emitted from the X-ray tube 11. More specifically, the wedge 16 is a filter configured to pass and attenuate the X-rays emitted from the X-ray tube 11 so that the X-rays emitted from the X-ray tube 11 onto the patient P has a predetermined distribution. The wedge 16 is a wedge filter or a bow-tie filter, for example, and is a filter obtained by processing aluminum so as to have a predetermined target angle and a predetermined thickness.

The collimator 17 is realized with lead plates or the like for narrowing down the X-rays that have passed through the wedge 16, into an X-ray emission range and is configured to form a slit with a combination of the plurality of lead plates or the like. The collimator 17 may be referred to as an X-ray limiter.

The Data Acquisition System (DAS) 18 includes a plurality of pieces of counting circuitry. Each of the plurality of pieces of counting circuitry includes an amplifier that performs an amplifying process on the electrical signals output from one or more of the detecting elements included in in the X-ray detector 12 and an Analog/Digital (A/D) converter that converts the amplified electrical signals into digital signals and is configured to generate the detection data, which is a result of a counting process using the detection signals from the X-ray detector 12. The result of the counting process is data in which an X-ray photon quantity is allocated for each energy bin. The energy bins correspond to energy bands each having a predetermined width. For example, the DAS 18 is configured to count the photons (X-ray photons) derived from the X-rays that were emitted from the X-ray tube 11 and have passed through the patient P and to generate the result of the counting process obtained by discriminating energy levels of the counted photons, as the detection data. The DAS 18 is an example of a data acquisition unit.

The detection data generated by the DAS 18 is transferred to the console apparatus 40. The detection data is a set of data indicating a channel number and a column number of a detector pixel at which the detection data was generated, a view number identifying an acquired view (which may be called “a projection angle”), and a value indicating the detected X-ray radiation amount. In this situation, as the view number, sequential order (an acquisition time) of the view acquisition may be used or a number (e.g., 1 to 1000) indicating a rotation angle of the X-ray tube 11 may be used. Each of the plurality of pieces of counting circuitry in the DAS 18 is realized, for example, by using a group of circuitry including circuitry elements capable of detecting the detection data. In the present embodiment, the simple term “detection data” inclusively denotes both pure raw data detected by the X-ray detector 12 on which pre-processing processes have not yet been performed and raw data obtained by performing the pre-processing processes on the pure raw data. In some situations, the data (the detection data) before the pre-processing processes and the data after the pre-processing processes may collectively be referred to as projection data.

The table apparatus 30 is an apparatus on which the patient P to be scanned is placed and moved and includes a base 31, a table driving apparatus 32, the tabletop 33, and a supporting frame 34. The base 31 is a casing configured to support the supporting frame 34 so as to be movable vertically. The table driving apparatus 32 is a motor or an actuator configured to move the tabletop 33 over which the patient P is placed in the longitudinal direction of the tabletop 33. The tabletop 33 provided on the top face of the supporting frame 34 is a board on which the patient P is placed. Further, in addition to the tabletop 33, the table driving apparatus 32 may be configured to move the supporting frame 34 in the longitudinal directions of the tabletop 33.

The console apparatus 40 includes a memory 41, a display 42, an input interface 43, and processing circuitry 44. Data communication among the memory 41, the display 42, the input interface 43, and the processing circuitry 44 is performed via a bus, for example. Although the console apparatus 40 is described as a separate apparatus from the gantry apparatus 10, the gantry apparatus 10 may include the console apparatus 40 or one or more of the constituent elements of the console apparatus 40.

For example, the memory 41 is realized by using a semiconductor memory element such as a Random Access Memory (RAM) or a flash memory, a Hard Disk Drive (HDD), a Solid State Drive (SSD), or an optical disc. Alternatively, the memory 41 may be a drive apparatus configured to read and write various types of information from and to a portable storage medium such as a Compact Disc (CD), a Digital Versatile Disc (DVD), or a flash memory, or a semiconductor memory element such as a Random Access Memory (RAN). For example, the memory 41 is configured to store therein the detection data output from the DAS 18, the projection data generated by the pre-processing function 442, and a reconstructed image reconstructed by a reconstruction processing function 443. For example, the reconstructed image may be three-dimensional CT image data (volume data) or two-dimensional CT image data. Further, the save area of the memory 41 may be provided within the PCCT apparatus 1 or within an external storage apparatus connected via a network.

The memory 41 is configured to store therein a trained model configured to generate, on the basis of first spectral data, second spectral data having less noise than the first spectral data and a higher resolution than the first spectral data. For example, the first spectral data corresponds to first pre-reconstruction data before being reconstructed or to a first reconstructed image, or the like. The first spectral data is medical data related to the patient P imaged by the spectral medical imaging apparatus. In other words, the first spectral data corresponds to the medical data obtained by performing a spectral scan on the patient P. The memory 41 is configured to store therein the first spectral data and the second spectral data generated (reconstructed) by the trained model.

When the first spectral data is the first pre-reconstruction data, while the spectral medical imaging apparatus is a DECT apparatus, the first pre-reconstruction data corresponds, for example, to first projection data acquired at first X-ray tube voltage by the DECT apparatus and to second projection data acquired at second X-ray tube voltage lower than the first X-ray tube voltage. In this situation, the first spectral data may be first reference projection data corresponding to each of two reference substances. Further, when the first spectral data is the first pre-reconstruction data, while the spectral medical imaging apparatus is the PCCT apparatus 1, the first pre-reconstruction data corresponds to first reference projection data corresponding to each of three or more reference substances or to first count data corresponding to each of a plurality of energy ranges.

When the first spectral data is the first reconstructed image, the first reconstructed image is one selected from among the following: a plurality of first reference substance images corresponding to a plurality of reference substances; at least one first virtual monochrome X-ray image having a different X-ray energy level; a first virtual non-contrast-enhanced image; a first iodine map image; a first effective atomic number image; a first electron density image; a first X-ray tube voltage image corresponding to the first X-ray tube voltage used in the imaging process performed by the spectral medical imaging apparatus and a second X-ray tube voltage image corresponding to the second X-ray tube voltage higher than the first X-ray tube voltage; and a plurality of first energy images corresponding to a plurality of energy ranges. The plurality of reference substances may be, for example, water, iodine, and/or the like. In that situation, the reference substance image may be, for example, a water image in which a water content amount (e.g., an abundance ratio of water) is expressed in each pixel or an iodine image in which an iodine content amount (e.g., an abundance ratio of iodine) is expressed in each pixel.

The first virtual monochrome X-ray image corresponds to a monochrome X-ray having a specific single energy component (keV) among the energy of the X-rays (e.g., white X-rays) generated by the X-ray tube 11 and represents a medical image such as that virtually taken by using a specific monochrome X-ray. The first virtual non-contrast-enhanced image corresponds to a first Virtual Non-Contrast (VNC) image generated from an contrast-enhanced image. The first iodine map image is a medical image indicating an extent of coloring by a contrast agent having iodine as a composition thereof. The first effective atomic number image is, for example, a medical image in which, with respect to the type(s) of element(s) in each of a plurality of voxels, the type of the element is indicated when a single element constitutes the voxel and an average atomic number is indicated when a plurality of elements constitute the voxel. In other words, the effective atomic number denotes a corresponding atomic number based on the assumption that a given voxel is substituted with a single atom. For example, the first effective atomic number image represents an image corresponding to a characteristic X-ray (k-edge) among the X-rays generated by the X-ray tube 11. The first electron density image is a medical image indicating the quantity of electrons estimated to be present within a unit volume. The first electron density image corresponds to a medical image indicating density of a contrast agent, for example. Each of the plurality of first energy images corresponds to a medical image generated on the basis of the detection data acquired by the PCCT apparatus 1 for a different one of the plurality of energy bins.

When the spectral medical imaging apparatus is a DECT apparatus, the first X-ray tube voltage image is a medical image reconstructed on the basis of the first projection data acquired at the first X-ray tube voltage by the DECT apparatus. Further, the second X-ray tube voltage image is a medical image reconstructed on the basis of the second projection data acquired at the second X-ray tube voltage higher than the first X-ray tube voltage.

The trained model is a model configured to realize a noise reducing process and a resolution increasing process on spectral data being input thereto and may be generated, for example, by training a convolution neural network (hereinafter, Deep Convolution Neural Network (DCNN)). Functions of the trained model include reconstructing processes in a broad sense. For this reason, the trained model in the present embodiment may be referred to as a deep learning reconstruction. The generation of the trained model (hereinafter, “noise-reduction super-resolution model”) in the present embodiment, i.e., the training of the DCNN, is realized by a training apparatus, any of various types of server apparatuses, any of various types of modalities in which the medical data processing apparatus is installed, or the like. For example, the generated noise-reduction super-resolution model is output from the apparatus that trained the DCNN and stored in the memory 41. The generation of the noise-reduction super-resolution model will be explained later.

For example, the second spectral data corresponds to second pre-reconstruction data corresponding to the first pre-reconstruction data or to a second reconstructed image corresponding to the first reconstructed image. The second spectral data is medical data related to the patient P imaged by the spectral medical imaging apparatus. The memory 41 is configured to store therein the second spectral data generated (reconstructed) by the trained model. When the first spectral data is the first projection data and the second projection data, the second spectral data is third projection data corresponding to the first projection data and fourth projection data corresponding to the second projection data. As another example, when the first spectral data is the first reference projection data, the second spectral data is second reference projection data corresponding to the first reference projection data. As yet another example, when the first spectral data is the first count data, the second spectral data is second count data corresponding to the first count data.

As yet another example, when the first reconstructed image is represented by the plurality of first reference substance images, the second reconstructed image is represented by a plurality of second reference substance images corresponding to the plurality of first reference substance images. As yet another example, when the first reconstructed image is the first virtual monochrome X-ray image, the second reconstructed image is a second virtual monochrome X-ray image corresponding to the first virtual monochrome X-ray image. When the first reconstructed image is the first virtual non-contrast-enhanced image, the second reconstructed image is a second virtual non-contrast-enhanced image corresponding to the first virtual non-contrast-enhanced image. When the first reconstructed image is the first iodine map image, the second reconstructed image is a second iodine map image corresponding to the first iodine map image. When the first reconstructed image is the first effective atomic number image, the second reconstructed image is a second effective atomic number image corresponding to the first effective atomic number image. When the first reconstructed image is the first electron density image, the second reconstructed image is a second electron density image corresponding to the first electron density image. When the first reconstructed image is represented by the plurality of first energy images, the second reconstructed image is represented by a plurality of second energy images corresponding to the plurality of first energy images. As yet another example, when the first reconstructed image is represented by the first X-ray tube voltage image and the second X-ray tube voltage image, the second reconstructed image is represented by a third X-ray tube voltage image corresponding to the first X-ray tube voltage image and a fourth X-ray tube voltage image corresponding to the second X-ray tube voltage image.

The memory 41 is configured to store therein programs related to implementing the system controlling function 441, the pre-processing function 442, the reconstruction processing function 443, an image processing function 444, and a data processing function 445 carried out by the processing circuitry 44. The memory 41 is configured to store therein the trained model compliant with a correspondence relationship between the first spectral data and the second spectral data. In an example, the memory 41 may store therein a plurality of trained models compliant with the correspondence relationship. The memory 41 is an example of a storage unit.

The display 42 is configured to display various types of information. For example, the display 42 is configured to output a medical image (a CT image) generated by the processing circuitry 44, a Graphical User Interface (GUI) used for receiving various types of operations from the operator, and the like. As the display 42, it is possible to use, for example, a Liquid Crystal Display (LCD), a Cathode Ray Tube (CRT) display, an Organic Electroluminescence Display (OELD), a plasma display, or any of other arbitrary displays, as appropriate. Further, the display 42 may be provided for the gantry apparatus 10. Also, the display 42 may be of a desktop type or may be configured by using a tablet terminal or the like capable of wirelessly communicating with the console apparatus 40 main body. The display 42 is an example of a display unit.

The input interface 43 is configured to receive various types of input operations from the operator, to convert the received input operations into electrical signals, and to output the electrical signals to the processing circuitry 44. For example, the input interface 43 is configured to receive, from the operator, an acquisition condition used at the time of acquiring the projection data, a reconstruction condition used at the time of reconstructing CT image data, an image processing condition related to a post-processing process on the CT image data, and the like. The post-processing process may be performed by the console apparatus 40 or by an external workstation. Alternatively, the post-processing process may simultaneously be performed by both the console apparatus 40 and the workstation. The post-processing process defined herein is a concept denoting a process performed on the image reconstructed by the reconstruction processing function 443. For example, examples of the post-processing process include displaying the reconstructed image according to a Multi Planar Reconstruction (MPR) scheme and volume data rendering. As the input interface 43, it is possible to use, for example, a mouse, a keyboard, a trackball, a switch, a button, a joystick, a touchpad, a touch panel display, and/or the like, as appropriate.

Further, in the present embodiment, the input interface 43 does not necessarily have to include physical operational component parts such as the mouse, the keyboard, the trackball, the switch, the button, the joystick, the touchpad, the touch panel display, and/or the like. For instance, possible examples of the input interface 43 include electrical signal processing circuitry configured to receive an electrical signal corresponding to an input operation from an external input mechanism provided separately from the apparatus and to output the electrical signal to the processing circuitry 44. Further, the input interface 43 is an example of an input unit. In an example, the input interface 43 may be provided for the gantry apparatus 10. Alternatively, the input interface 43 may be configured by using a tablet terminal or the like capable of wirelessly communicating with the console apparatus 40 main body.

The processing circuitry 44 is configured, for example, to control operations of the entirety of the PCCT apparatus 1 in accordance with the electrical signals of the input operations output from the input interface 43. For example, the processing circuitry 44 includes, as hardware resources thereof, a processor such as a CPU, an MPU, or a Graphics Processing Unit (GPU) and one or more memory elements such as a ROM, a RAM, and/or the like. By employing the processor that executes the programs loaded into any of the memory elements of the processing circuitry 44, the processing circuitry 44 is configured to carry out the system controlling function 441, the pre-processing function 442, the reconstruction processing function 443, the image processing function 444, and the data processing function 445. The functions 441 to 445 do not necessarily need to be realized by the single piece of processing circuitry. It is also acceptable to structure processing circuitry by combining together a plurality of independent processors, so that the functions 441 to 445 are realized as a result of the processors executing the programs.

The system controlling function 441 is configured to control the functions of the processing circuitry 44 on the basis of the input operations received from the operator via the input interface 43. Further, the system controlling function 441 is configured to read a control program stored in the memory 41, to load the read control program into a memory element in the processing circuitry 44, and to control functional units of the PCCT apparatus 1 according to the loaded control program. The system controlling function 441 is an example of a controlling unit.

The pre-processing function 442 is configured to generate the projection data obtained by performing pre-processing processes such as a logarithmic conversion process, an offset correction process, an inter-channel sensitivity correction process, a beam hardening correction, and/or the like, on the detection data output from the DAS 18. Because the generating process of the first pre-reconstruction data (e.g., the first projection data, the second projection data, a plurality of pieces of first reference projection data, and a plurality of pieces of first count data) generated by the pre-processing function 442 is compliant with known processing procedures, explanations thereof will be omitted. The pre-processing function 442 is an example of a pre-processing unit.

The reconstruction processing function 443 is configured to generate the CT image data by performing a reconstructing process that uses a Filtered Back Projection (FBP) method or the like, on the projection data generated by the pre-processing function 442. The reconstructing process includes various types of processes such as various types of correcting processes including a scattering property correction and a beam hardening correction, and application of a reconstruction mathematical function in the reconstruction condition. Further, to the reconstructing process performed by the reconstruction processing function 443, it is acceptable to apply not only the FBP method, but also any of known processes, as appropriate, such as a successive approximation reconstruction or a deep neural network configured to receive an input of the projection data and to output a reconstructed image. The reconstruction processing function 443 is configured to store the reconstructed CT image data into the memory 41. The reconstructing process realized by the reconstruction processing function 443 is not limited to generating an image on the basis of the pre-reconstruction data such as the projection data, but has a function of realizing the reconstructing processes in a broad sense. For example, the reconstruction processing function 443 is configured, on the basis of the first pre-reconstruction data, to generate the first reconstructed image (the plurality of first reference substance images, the first virtual monochrome X-ray image, the first VNC image, the first iodine map image, the first effective atomic number image, the first electron density image, the first X-ray tube voltage image, the second X-ray tube voltage image, the plurality of first energy images, or the like). Because the process of generating the first reconstructed image is compliant with known processing procedures, explanations thereof will be omitted. The reconstruction processing function 443 is an example of a reconstruction processing unit.

The image processing function 444 is configured, on the basis of an input operation received from the operator via the input interface 43, to convert the CT image data generated by the reconstruction processing function 443 into tomographic image data on an arbitrary cross-sectional plane or three-dimensional image data by using a publicly-known method. Alternatively, the process of generating the three-dimensional image data may directly be performed by the reconstruction processing function 443. Further, the image processing function 444 is an example of an image processing unit.

The data processing function 445 is configured to output, through the noise-reduction super-resolution model, the second spectral data realizing less noise and a higher resolution than the first spectral data, by inputting the first spectral data to the noise-reduction super-resolution model serving as the trained model. In other words, the trained model is configured to perform processes of reducing the noise and increasing the resolution (a noise reducing process and a super-resolution process) on the first spectral data. The super-resolution corresponds to increasing the resolution of the data. For example, the data processing function 445 is configured to input the first pre-reconstruction data to the noise-reduction super-resolution model and to cause the noise-reduction super-resolution model to output the second pre-reconstruction data realizing less noise and a higher resolution than the first pre-reconstruction data. In that situation, the second pre-reconstruction data realizing the less noise and the higher resolution is reconstructed by the reconstruction processing function 443, so as to generate the second reconstructed image having less noise and a higher resolution compared to the first reconstructed image reconstructed on the basis of the first pre-reconstruction data.

In another example, when the input (the first spectral data) to the noise-reduction super-resolution model is the first reconstructed image reconstructed on the basis of raw data acquired from the imaging process performed on the patient P by the spectral medical imaging apparatus, the data processing function 445 is configured to input the first reconstructed image to the noise-reduction super-resolution model and to cause the noise-reduction super-resolution model to output the second reconstructed image realizing less noise and a higher resolution than the first reconstructed image, as the second spectral data. The second reconstructed image is a medical image which corresponds to the image type of the first reconstructed image while having less noise than the first reconstructed image and of which the noise has been reduced and the resolution has been increased compared to the first reconstructed image.

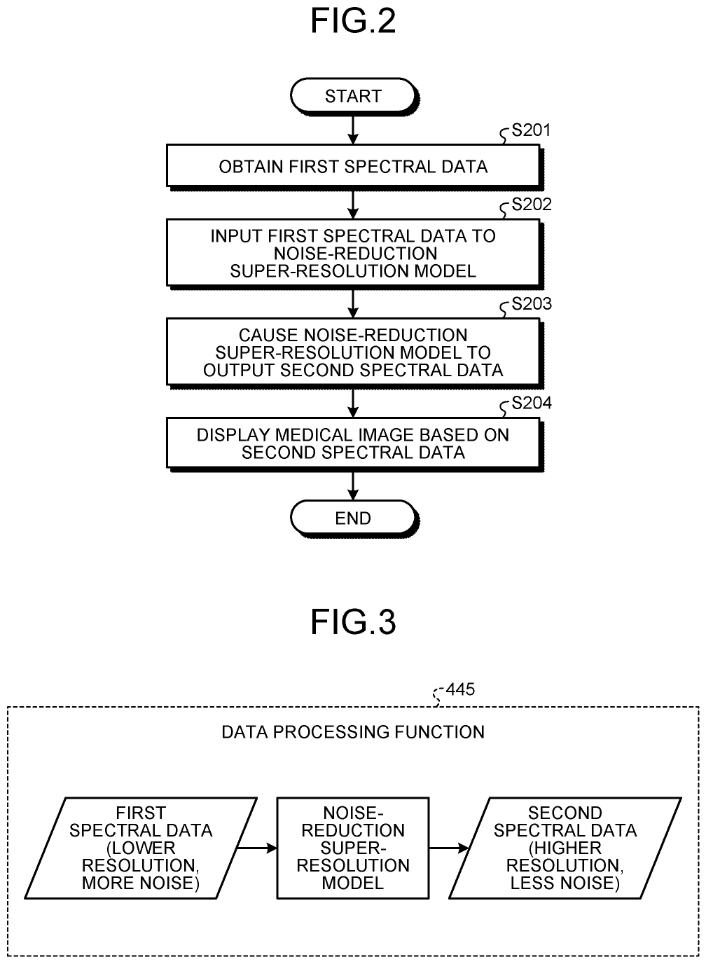

A process (hereinafter, “noise-reduction super-resolution process”) performed by the PCCT apparatus 1 according to the present embodiment configured as described above, to generate the second spectral data from the first spectral data by employing the noise-reduction super-resolution model will be explained, with reference to FIGS. 2 to 6.

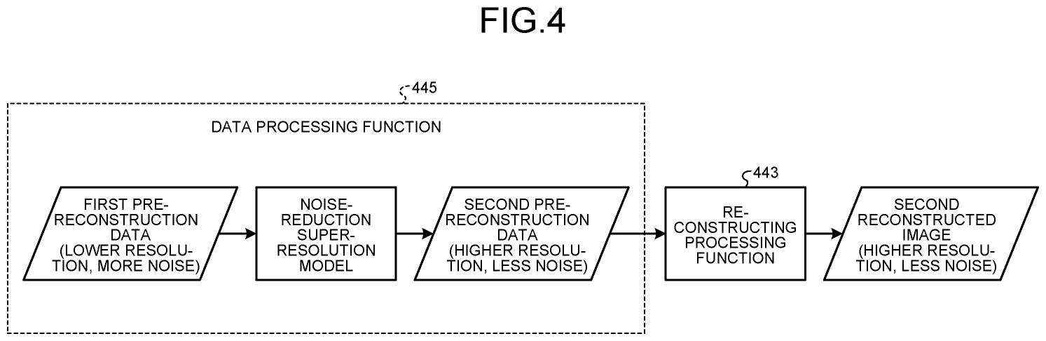

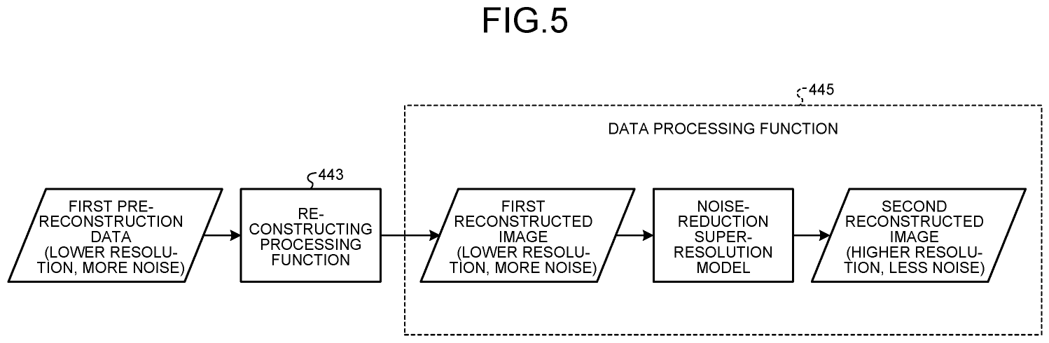

FIG. 2 is a flowchart illustrating an example of a procedure in the noise-reduction super-resolution process. FIG. 3 is a diagram illustrating an outline of the noise-reduction super-resolution process using the first spectral data. FIG. 4 is a diagram illustrating an outline of a noise-reduction super-resolution process using the projection data as an example of the first spectral data. FIG. 5 is a diagram illustrating an outline of a noise-reduction super-resolution process using a reconstructed image as an example of the first spectral data.

### The Noise-Reduction Super-Resolution Process

### Step S201:

By employing the data processing function 445, the processing circuitry 44 obtains the first spectral data to be input to the noise-reduction super-resolution process. For example, when the spectral medical imaging apparatus is a DECT apparatus, the data processing function 445 obtains, from the pre-processing function 442, the first projection data (a lower resolution and more noise) and the second projection data (a lower resolution and more noise) generated by scanning the patient P with a low radiation dose. In another example, when the spectral medical imaging apparatus is a DECT apparatus or the PCCT apparatus 1, the data processing function 445 obtains, for example, the first reference projection data (a lower resolution and more noise) from the pre-processing function 442. In yet another example, when the spectral medical imaging apparatus is the PCCT apparatus 1, the data processing function 445 obtains, for example, a plurality of pieces of first count data (a lower resolution and more noise) generated by scanning the patient P, from the pre-processing function 442.

In yet another example, when the spectral medical imaging apparatus is a DECT apparatus, the data processing function 445 obtains, for example, the first X-ray tube voltage image (a lower resolution and more noise) and the second X-ray tube voltage image (a lower resolution and more noise) from the reconstruction processing function 443. In yet another example, when the spectral medical imaging apparatus is a DECT apparatus or the PCCT apparatus 1, the data processing function 445 obtains, for example, obtains one (a lower resolution and more noise) of the following images from the reconstruction processing function 443: the plurality of first reference substance images, the first virtual monochrome X-ray image, the first VNC image, the first iodine map image, the first effective atomic number image, and the first electron density image. In yet another example, when the spectral medical imaging apparatus is the PCCT apparatus 1, the data processing function 445 obtains, for example, the plurality of energy images (a lower resolution and more noise) from the reconstruction processing function 443.

In yet another example, when the noise-reduction super-resolution process is performed by the medical data processing apparatus, the data processing function 445 obtains data to be input to the noise-reduction super-resolution model in the noise-reduction super-resolution process, from a medical image taking apparatus or an image storage server of an image saving communication system (e.g., a Picture Archiving and Communication System; hereinafter, “PACS”).

When the execution of the noise-reduction super-resolution process is turned off, i.e., when the trained model (the noise-reduction super-resolution model) is not in use, the reconstruction processing function 443 is configured to reconstruct a first reconstructed image having a first matrix size, on the basis of the acquisition data (the first pre-reconstruction data) acquired from the imaging process performed on the patient P by the spectral medical imaging apparatus, by employing, for example, a known deep-learning trained CNN (hereinafter, “noise reduction model”) that performs only a noise reducing process, or the like. The first matrix size may be a matrix size of 512×512, for example. In contrast, when the noise-reduction super-resolution process is turned on, i.e., when the trained model (the noise-reduction super-resolution model) is in use, the reconstruction processing function 443 is configured to reconstruct a first reconstructed image on the basis of the first pre-reconstruction data, so as to have a second matrix size which is larger than the first matrix size and corresponds to the matrix size of the second reconstructed image. The second matrix size may be a matrix size of 1024×1024 or a matrix size of 2048×2048, for example. In this situation, by inputting the first reconstructed image having the second matrix size to the trained model at step S202 (explained later), the data processing function 445 outputs the second reconstructed image at step S203 (explained later).

Further, when the noise-reduction super-resolution process is turned off at the time of scanning the patient P, a first reconstructed image is generated in the first matrix size. After that, when the noise-reduction super-resolution process is turned on according to an instruction from the operator received via the input interface 43, the data processing function 445 is configured to perform up-sampling on the first reconstructed image to change the first matrix size into the second matrix size. In other words, at the point in time when the noise-reduction super-resolution process is turned on, if the first matrix size is smaller than the second matrix size, the data processing function 445 up-samples the first matrix size into the second matrix size. In this situation, the data processing function 445 is configured to input the first reconstructed image having the second matrix size to the trained model so as to output the second reconstructed image. Further, when the noise-reduction super-resolution process is turned on after a first reconstructed image is generated in the first matrix size, the reconstruction processing function 443 may, again, reconstruct another first reconstructed image having the second matrix size as the first spectral data, on the basis of the first pre-reconstruction data.

### Step S202:

The data processing function 445 reads the noise-reduction super-resolution model from the memory 41. For example, the data processing function 445 reads the noise-reduction super-resolution model corresponding to the type of the first spectral data, from the memory 41. For example, when a plurality of pieces of count data corresponding to the plurality of energy bins are used as the first spectral data, the data processing function 445 reads a plurality of noise-reduction super-resolution models corresponding to the plurality of energy bins, from the memory 41. The data processing function 445 inputs the first spectral data to the noise-reduction super-resolution model. For example, when the first pre-reconstruction data is used as the first spectral data, the data processing function 445 inputs the first pre-reconstruction data (a lower resolution and more noise) to the noise-reduction super-resolution model. In another example, when the first reconstructed image is used as the first spectral data, the data processing function 445 inputs the first reconstructed image (a lower resolution and more noise) to the noise-reduction super-resolution model.

### Step S203:

The data processing function 445 causes the noise-reduction super-resolution model to output the second spectral data. For example, when the first pre-reconstruction data is input to the noise-reduction super-resolution model, the data processing function 445 causes the noise-reduction super-resolution model to output the second pre-reconstruction data having a higher resolution and less noise. In this situation, the reconstruction processing function 443 generates the second reconstructed image on the basis of the second pre-reconstruction data having a higher resolution and less noise. Further, when the first reconstructed image is input to the noise-reduction super-resolution model, the data processing function 445 causes the noise-reduction super-resolution model to output the second reconstructed image having a higher resolution and less noise.

### Step S204:

The system controlling function 441 causes the display 42 to display a medical image based on the second spectral data. In an example, according to an instruction from the operator received via the input interface 43, the image processing function 444 may perform any of various types of image processing processes on the medical image. In that situation, the system controlling function 441 causes the display 42 to display the medical image to which the image processing processes have been applied.

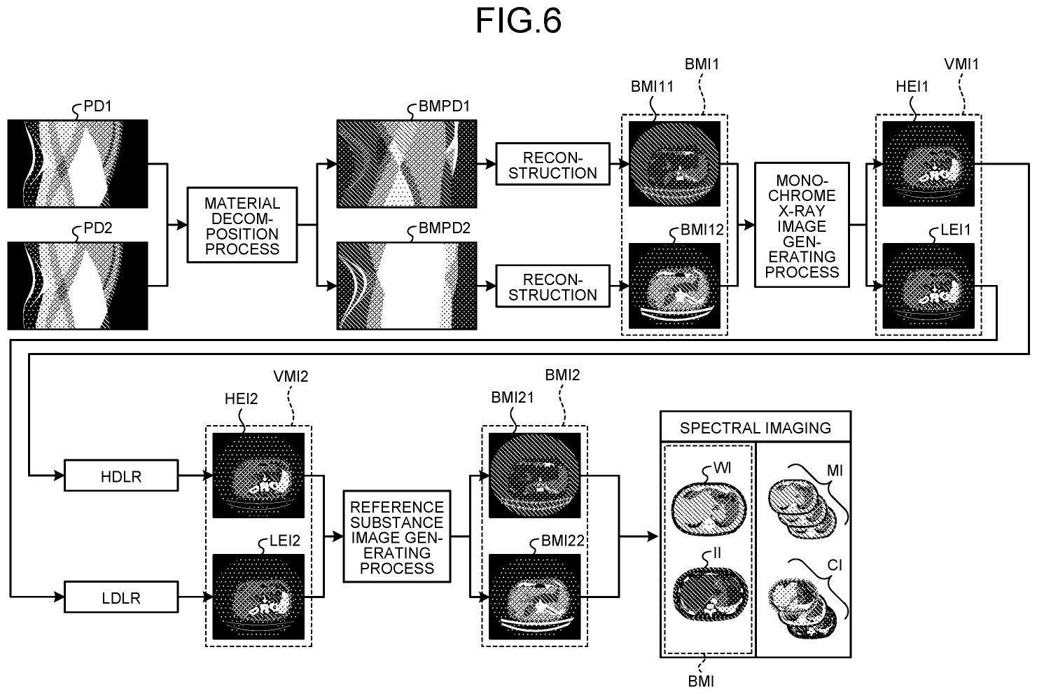

FIG. 6 is a diagram illustrating an example in which the noise-reduction super-resolution process is applied to the first reconstructed image generated by a DECT apparatus. As illustrated in FIG. 6, the DECT apparatus is configured to generate first projection data PD1 by performing a scan using the first X-ray tube voltage and to generate second projection data PD2 by performing a scan using the second X-ray tube voltage. Subsequently, the data processing function 445 (or the reconstruction processing function 443) performs a material decomposition process on the first projection data PD1 and the second projection data PD2, to generate raw data BMPD1 of a first reference substance and raw data BMPD2 of a second reference substance. Alternatively, the material decomposition process may be performed by the image processing function 444. Further, because any of known techniques is applicable to the material decomposition process, explanations thereof will be omitted. For example, it is possible to use a method disclosed in Japanese Patent Application Laid-open No. 2009-261942 or a method using a neural network disclosed in the specification of US patent application No. 2015/371378.

As illustrated in FIG. 6, the reconstruction processing function 443 performs a reconstructing process on the raw data BMPD1 of the first reference substance and the raw data BMPD2 of the second reference substance, to generate a first reference substance image BMI11 corresponding to a first substance and a second reference substance image BMI12 corresponding to a second substance. The first reference substance image BMI11 corresponding to the first substance and the second reference substance image BMI12 corresponding to the second substance are first reference substance images BMI1 corresponding to the two types of reference substances.

As illustrated in FIG. 6, the data processing function 445 (or the image processing function 444) performs a monochrome X-ray image generating process on the first reference substance image BMI11 corresponding to the first substance and the second reference substance image BMI12 corresponding to the second substance, to generate first virtual monochrome X-ray images VMI1 having mutually-different energy levels. The first virtual monochrome X-ray images VMI1 include a virtual monochrome X-ray image having relatively higher energy (hereinafter, “first higher energy monochrome image”) HEI1 and a virtual monochrome X-ray image having relatively lower energy (hereinafter, “first lower energy monochrome image”) LEI1. The first virtual monochrome X-ray images VMI1 correspond to the first reconstructed image. Alternatively, the monochrome X-ray image generating process may be performed by the reconstruction processing function 443. Further, because any of known technique is applicable to the monochrome X-ray image generating process, explanations thereof will be omitted.

As illustrated in FIG. 6, the data processing function 445 reads, from the memory 41, two types of noise-reduction super-resolution models corresponding to the first higher energy monochrome image HEI1 and the first lower energy monochrome image LEI1. As illustrated in FIG. 6, the two types of noise-reduction super-resolution models are, namely, a noise-reduction super-resolution model (hereinafter, High energy Deep Learning Reconstruction “HDLR”) corresponding to the energy and the image type of the higher energy monochrome image HEI1 and a noise-reduction super-resolution model (hereinafter, Low energy Deep Learning Reconstruction “LDLR”) corresponding to the energy and the image type of the lower energy monochrome image LEI1.

As illustrated in FIG. 6, the data processing function 445 inputs the first higher energy monochrome image HEI1 to the High energy Deep Learning Reconstruction HDLR, to generate a second higher energy monochrome image HEI2 realizing less noise and a higher resolution than the first higher energy monochrome image HEI1. Further, the data processing function 445 inputs the first lower energy monochrome image LEI1 to the Low energy Deep Learning Reconstruction LDLR, to generate a second lower energy monochrome image LEI2 realizing less noise and a higher resolution than the first lower energy monochrome image LEI1. The second higher energy monochrome image HEI2 and the second lower energy monochrome image LEI2 are second virtual monochrome X-ray images VMI2 and correspond to the second reconstructed image.

As illustrated in FIG. 6, the data processing function 445 (or the image processing function 444) performs a reference substance image generating process on the second virtual monochrome X-ray images VMI2 (the second higher energy monochrome image HEI2 and the second lower energy monochrome image LEI2), to generate second reference substance images BMI2 realizing less noise and a higher resolution. The second reference substance images BMI2 include a first reference substance image BMI21 realizing less noise and a higher resolution than the first reference substance image BMI11 and a second reference substance image BMI22 realizing less noise and a higher resolution than the second reference substance image BMI12. Alternatively, the reference substance image generating process may be performed by the reconstruction processing function 443. Further, because any of known techniques is applicable to the reference substance image generating process, explanations thereof will be omitted.

The data processing function 445 (or the image processing function 444) performs, as illustrated in FIG. 6, spectral imaging on the second reference substance image BMI12. The spectral imaging is image processing related to spectral data. In an example, the term “spectral imaging” may be used in a sense including a spectral scan and image processing. By performing the spectral imaging related to the image processing, the data processing function 445 (or the image processing function 444) generates, as illustrated in FIG. 6, substances images related to a plurality of substances (e.g., a substance image WI related to water and a substance image Il related to iodine) BMI, the virtual monochrome X-ray images MI corresponding to the plurality of energy levels, and a composite image CI obtained by combining together various types of images.

Although the example was explained with reference to FIG. 6 in which the first spectral data input to the trained model is the first virtual monochrome X-ray images VMI1; however, possible embodiments are not limited to this example. In other words, the first spectral data may be the first projection data PD1 and the second projection data PD2; the raw data BMPD1 of the first reference substance and the raw data BMPD2 of the second reference substance; or the first reference substance images MI1. In that situation, the data processing function 445 is configured to read a noise-reduction super-resolution model corresponding to the type of the first spectral data from the memory 41 and to perform the noise-reduction super-resolution process by inputting the first spectral data to the read noise-reduction super-resolution model.

The spectral medical imaging apparatus (e.g., the DECT apparatus or the PCCT apparatus 1) according to the embodiment described above is configured to output the second spectral data by inputting the first spectral data related to the patient P imaged by the spectral medical imaging apparatus, to the trained model that, on the basis of the first spectral data, generates the second spectral data having less noise than the first spectral data and a more high resolution than the first spectral data. In the present medical data processing apparatus, the first spectral data corresponds to the medical data obtained from the spectral scan performed on the patient P, and the trained model is configured to process the first spectral data.

Further, in the spectral medical imaging apparatus according to the embodiment, the first spectral data may be, for example, the first pre-reconstruction data before being reconstructed that was acquired from the imaging process performed on the patient P by the spectral medical imaging apparatus, whereas the second spectral data may be the second pre-reconstruction data before being reconstructed, so that the medical image is generated on the basis of the second pre-reconstruction data before being reconstructed. In that situation, in the spectral medical imaging apparatus according to the embodiment, for example, the first pre-reconstruction data corresponds to the first projection data PD1 acquired at the first X-ray tube voltage by the spectral medical imaging apparatus and the second projection data PD2 acquired at the second X-ray tube voltage higher than the first X-ray tube voltage, whereas the second pre-reconstruction data corresponds to the third projection data corresponding to the first projection data and to the fourth projection data corresponding to the second projection data. In another example, in the spectral medical imaging apparatus according to the embodiment, the first pre-reconstruction data may be the first reference projection data (e.g., the raw data BMPD1 of the first reference substance and the raw data BMPD2 of the second reference substance) corresponding to each of the plurality of reference substances, whereas the second pre-reconstruction data may be the second reference projection data corresponding to the first reference projection data. In yet another example, in the spectral medical imaging apparatus according to the embodiment, for instance, the first pre-reconstruction data may be the first count data corresponding to each of the plurality of energy ranges, whereas the second pre-reconstruction data may be the second count data corresponding to the first count data.

In another example, in the spectral medical imaging apparatus according to the embodiment, for instance, the first spectral data may be the first reconstructed image reconstructed on the basis of the acquisition data acquired from the imaging process performed on the patient P by the spectral medical imaging apparatus, whereas the second spectral data may be the second reconstructed image having less noise than the first reconstructed image and a more super resolution than the first reconstructed image. In that situation, in the spectral medical imaging apparatus according to the embodiment, for example, the first reconstructed image may be represented by the plurality of first reference substance images corresponding to the plurality of reference substances, whereas the second reconstructed image may be represented by the plurality of second reference substance images corresponding to the plurality of first reference substance images. In yet another example, in the spectral medical imaging apparatus according to the embodiment, for instance, the first reconstructed image may be one or more first virtual monochrome X-ray images VMI1 having mutually-different X-ray energy levels (e.g., the first higher energy monochrome image HEI1 and the first lower energy monochrome image LEI1), whereas the second reconstructed image may be the second virtual monochrome X-ray images VMI2 (e.g., the second higher energy monochrome image HEI2 and the second lower energy monochrome image LEI2) corresponding to the first virtual monochrome X-ray images VMI1. In yet another example, in the spectral medical imaging apparatus according to the embodiment, for instance, the first reconstructed image may be the first virtual non-contrast-enhanced image, whereas the second reconstructed image may be the second virtual non-contrast-enhanced image corresponding to the first virtual non-contrast-enhanced image.

In yet another example, in the spectral medical imaging apparatus according to the embodiment, for instance, the first reconstructed image may be the first iodine map image, whereas the second reconstructed image may be the second iodine map image corresponding to the first iodine map image. In yet another example, in the spectral medical imaging apparatus according to the embodiment, for instance, the first reconstructed image may be the first effective atomic number image, whereas the second reconstructed image may be the second effective atomic number image corresponding to the first effective atomic number image. In yet another example, in the spectral medical imaging apparatus according to the embodiment, for instance, the first reconstructed image may be the first electron density image, whereas the second reconstructed image may be the second electron density image corresponding to the first electron density image. In yet another example, in the spectral medical imaging apparatus according to the embodiment, for instance, the first reconstructed image may be represented by the plurality of first energy images corresponding to the plurality of energy ranges, whereas the second reconstructed image may be represented by the plurality of second energy images corresponding to the plurality of first energy images. In yet another example, in the spectral medical imaging apparatus according to the embodiment, the first reconstructed image may be represented by the first X-ray tube voltage image corresponding to the first X-ray tube voltage used in the imaging performed by the spectral medical imaging apparatus and the second X-ray tube voltage image corresponding to the second X-ray tube voltage higher than the first X-ray tube voltage, whereas the second reconstructed image may be represented by the third X-ray tube voltage image corresponding to the first X-ray tube voltage image and the fourth X-ray tube voltage image corresponding to the second X-ray tube voltage image.

As explained above, by employing the trained model corresponding to the type (e.g., the first pre-reconstruction data (i.e., the first projection data, the second projection data, the first reference projection data, the first count data, etc.) and the first reconstructed image (i.e., the first reference substance image, the first virtual monochrome X-ray image, the first virtual non-contrast-enhanced image, the first iodine map image, the first effective atomic number image, the first electron density image, the first X-ray tube voltage image and the second X-ray tube voltage image, the first energy image, etc.)) of the first spectral data obtained by the spectral medical imaging apparatus, the spectral medical imaging apparatus according to the present embodiment is able to realize, at the same time, both enhancing the spatial resolution (the resolution increasing process: super-resolution) and reducing the noise (the noise reducing process) of the first spectral data. Consequently, the spectral medical imaging apparatus according to the present embodiment is able to generate the medical image in which visibility is enhanced for objects such as anatomical characteristics in the medical image, while the image quality thereof is also enhanced. As a result, the spectral medical imaging apparatus according to the present embodiment is able to reduce radiation exposure of the patient P and to also enhance throughput of image diagnosis processes related to the patient P.

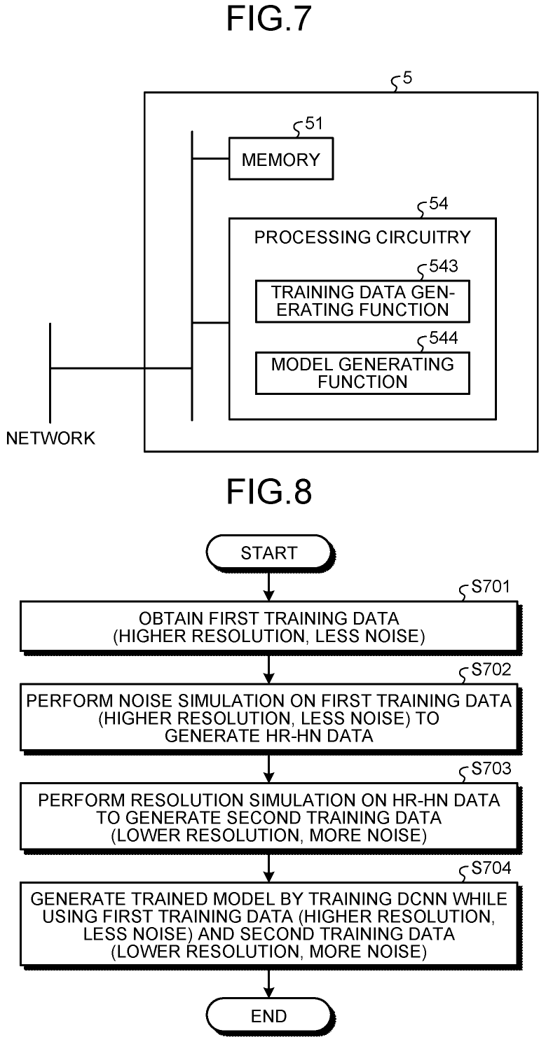

Next, a process of generating (a model generating method for) the trained model (the noise-reduction super-resolution model) used in the embodiment will be explained. FIG. 7 is a diagram illustrating an exemplary configuration of a training apparatus 5 related to generating the noise-reduction super-resolution model. In an example, the function of realizing the training of the DCNN by the training apparatus 5 may be installed in a medical image taking apparatus such as the spectral medical imaging apparatus or a medical data processing apparatus. For example, when the trained model is stored in the PCCT apparatus 1, the training apparatus 5 is configured to train the DCNN in accordance with a setting of energy bins corresponding to a scan plan of the PCCT apparatus 1. For example, when the quantity of the energy bins being set is four in a specific scan plan, the training apparatus 5 is configured to train the DCNN with respect to each of the four energy bins.

The memory 51 is configured to store therein a pair of training data sets generated by a training data generating function 543 of processing circuitry 54. Further, the memory 51 is configured to store therein source data from which the training data is generated. The source data may be obtained, for example, from the spectral medical imaging apparatus related to the data to be processed by the noise-reduction super-resolution model. Further, the memory 51 is configured to store therein the DCNN to be trained and the trained model (the noise-reduction super-resolution model). The memory 51 is configured to store therein programs related to implementing the training data generating function 543 and a model generating function 544 carried out by the processing circuitry 54. The memory 51 is an example of a storage unit for the training apparatus 5. Further, because the hardware and the like realizing the memory 51 are the same as those of the memory 41 described in the embodiment, explanations thereof will be omitted.

By employing a processor that executes the programs loaded into a memory of the processing circuitry 54, the processing circuitry 54 is configured to carry out the training data generating function 543 and the model generating function 544. Because the hardware and the like realizing the processing circuitry 54 are the same as those of the processing circuitry 44 described in the embodiment, explanations thereof will be omitted.

The training data generating function 543 is configured to obtain first training data corresponding to the noise and the resolution of the second spectral data. The training data generating function 543 is configured to generate second training data corresponding to the noise and the resolution of the first spectral data, by adding noise to and reducing the resolution of the first training data (a noise adding process and a resolution lowering process). For example, by performing a noise simulation, the training data generating function 543 is configured to add the noise to the first training data. Subsequently, by performing a resolution simulation, the training data generating function 543 is configured to lower the resolution of the first training data to which the noise has been added. The order in which the noise simulation and the resolution simulation are performed on the first training data is not limited to the order explained above and may be the reverse order. Further, because any of known techniques may be used for the noise simulation and the resolution simulation, explanations thereof will be omitted.

As a result, the training data generating function 543 has obtained the second training data forming a pair with the first training data. The first training data corresponds to teacher data (correct answer data) for the second training data. The training data generating function 543 is configured to store the generated first training data and second training data into the memory 51. By repeatedly performing the above process, the training data generating function 543 is configured to generate a plurality of training data sets in each of which first training data is paired with second training data and to store the generated training data sets into the memory 51.

The model generating function 544 is configured to generate the trained model by training the convolution neural network by using the first training data and the second training data. In other words, the model generating function 544 is configured to train the DCNN by applying the first training data and the second training data to the DCNN to be trained and to thus generate the noise-reduction super-resolution model.

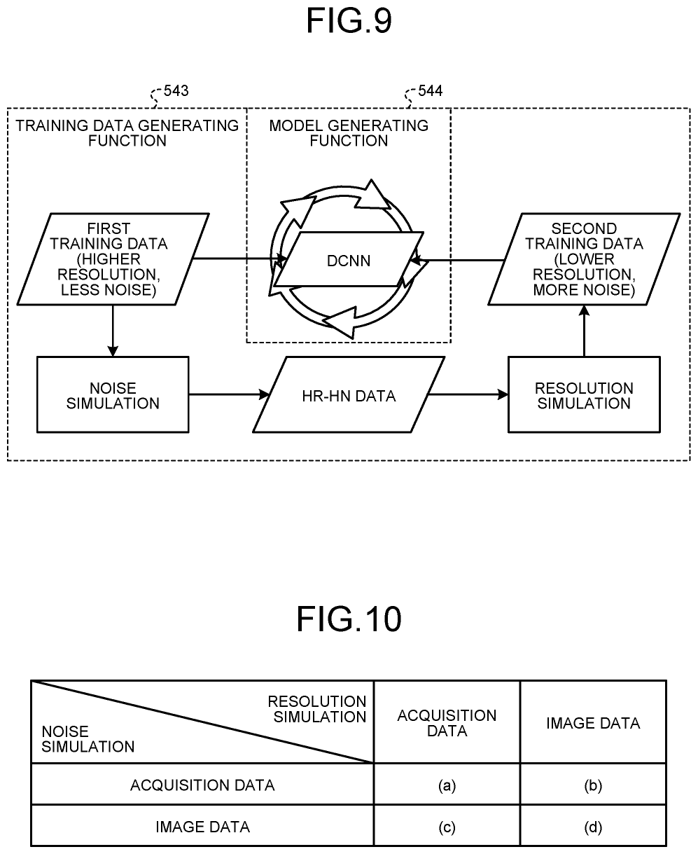

FIG. 8 is a flowchart illustrating an exemplary procedure in a process (hereinafter, “model generating process”) to generate the noise-reduction super-resolution model by training the DCNN while using the first training data and the second training data. FIG. 9 is a diagram illustrating an outline of the model generating process.

### The Model Generating Process

### Step S701:

The training data generating function 543 obtains the first training data. For example, the training data generating function 543 obtains, as the first training data, data acquired from a high resolution mode imaging process performed by a spectral medical imaging apparatus capable of acquiring medical data having a higher spatial resolution than a spectral medical imaging apparatus of a normal resolution and capable of imaging the patient P or performed by a high resolution spectral medical imaging apparatus including an X-ray detector (hereinafter, “high resolution detector”) having a higher spatial resolution, from one of these apparatuses. The high resolution mode corresponds to acquiring data from each of a plurality of X-ray detecting elements in the high resolution detector. In this situation, acquisition of the second spectral data by the high resolution spectral medical imaging apparatus corresponds, for example, to acquiring an average of outputs from four X-ray detecting elements that are positioned adjacent to each other among the plurality of X-ray detecting elements in the high resolution detector. The training data generating function 543 saves the first training data into the memory 51.

### Step S702:

The training data generating function 543 performs the noise simulation on the first training data to generate data (hereinafter, “HR-HN data”) having a high resolution (HR) and high noise (HN). The HR-HN data has more noise than the first training data does. In other words, the first training data corresponds to high-resolution low-noise (HR-LN) data having a lower level of noise (LN) than the HR-HN data. The noise in the HR-HN data corresponds to the noise level of first medical data, for example.

For example, the noise simulation may implement a method by which noise based on a predetermined statistic model such as Gaussian noise is added to the first training data or may implement a method by which noise based on a noise model trained in advance in relation to one or more detection systems such as the DAS 18 and/or the X-ray detector is added to the first training data. Because these methods are known, explanations thereof will be omitted. Further, the noise simulation is not limited to the methods described above and may be realized by any of other known methods.

### Step S703:

The training data generating function 543 performs the resolution simulation on the HR-HN data to generate data (hereinafter, “LR-HN data”) having a low resolution (LR) and high noise (HN), as the second training data. The LR-HN data has a lower resolution than the first training data. The resolution of the LR-HN data corresponding to the second training data corresponds to the resolution of the first medical data, for example.

The resolution simulation may implement, for example, a down-sampling and/or up-sampling method such as bi-cubic, bi-linear, box, or neighbor; a method using a smoothing filter and/or a sharpening filter; a method using a prepared model such as a Point Spread Function (PSF); or a down-sampling process simulating an acquisition data system by acquiring an average of outputs from four X-ray detecting elements that are positioned adjacent to each other, for example, among the plurality of X-ray detecting elements in the high resolution detector of the spectral medical imaging apparatus. Because these methods are known, explanations thereof will be omitted. Further, the resolution simulation is not limited to the methods described above and may be realized by using any of other known methods.

Although the procedure was explained above in which, after the noise simulation is performed on the first training data, the resolution simulation is performed, possible embodiments are not limited to this example. For instance, the training data generating function 543 may generate data (hereinafter, “LR-LN data”) having a lower resolution and less noise by performing a resolution simulation on the first training data and may subsequently perform a noise simulation on the LR-LN data so as to generate the second training data (LR-HN data).

By repeatedly performing the processes at steps S701 through S703, the training data generating function 543 generates the plurality of training data sets in each of which first training data is paired with second training data. The training data generating function 543 stores the generated plurality of training data sets into the memory 51. Alternatively, the training data generating process may be performed repeatedly after the process on the subsequent stage at step S704, until the training of the DCNN converges.

### Step S704

The model generating function 544 trains the DCNN by applying the first training data and the second training data to the DCNN to be trained. Because any of known methods such as a gradient descent method is applicable to the training process of the DCNN performed by the model generating function 544 while using the plurality of training data sets, explanations thereof will be omitted. As being triggered by convergence of the training of the DCNN, the model generating function 544 stores the trained DCNN into the memory 51 as a noise-reduction super-resolution model. The noise-reduction super-resolution model stored in the memory 51 is, for example, transmitted to the medical image taking apparatus related to the first training data and/or a medical data processing apparatus that implements the noise-reduction super-resolution model, as appropriate.

Next, an example of the data subject to the noise simulation and the resolution simulation will be explained. FIG. 10 is a table illustrating examples of combinations of the data subject to the noise simulation and the resolution simulation. The acquisition data (the second spectral data) in FIG. 10 may vary in accordance with the type of the spectral medical imaging apparatus or the like. The acquisition data, for example, may be the third projection data and the fourth projection data for a DECT apparatus or the like, may be the second reference projection data for a DECT apparatus or the PCCT apparatus 1, and may be the second count data for the PCCT apparatus 1. In the following sections, to explain specific examples, the acquisition data will be assumed to be the third projection data. Further, the image data in FIG. 10 is, for example, the second reconstructed image described above.

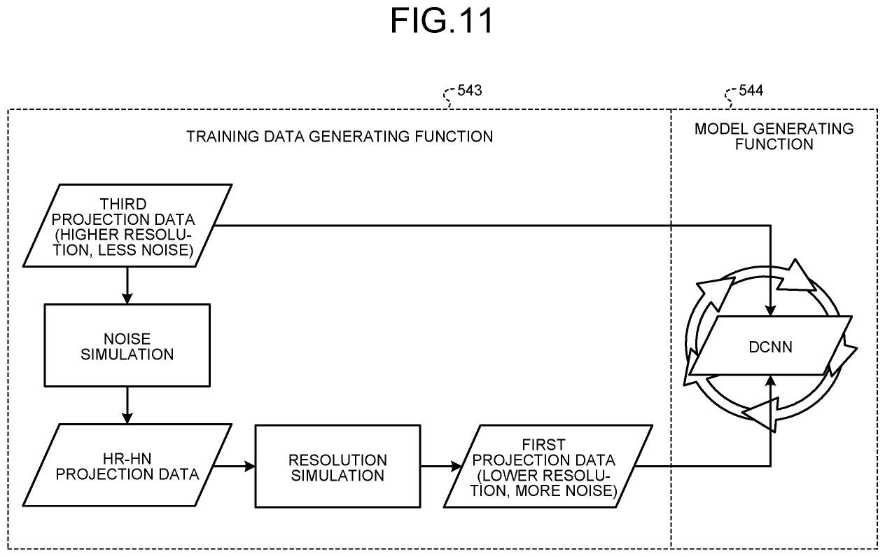

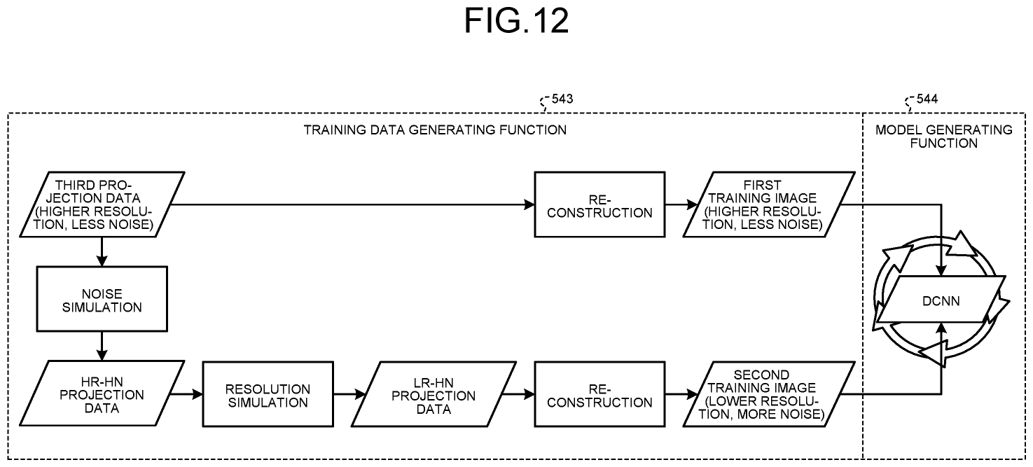

First, FIG. 10 (a) will be explained, with reference to FIGS. 11 and 12. FIG. 11 is a diagram related to FIG. 10 (a) illustrating an outline of a model generating process in the situation where projection data is an input/output of a noise-reduction super-resolution model serving as the trained model. FIG. 12 is a diagram related to FIG. 10 (a) illustrating an outline of a model generating process in the situation where image data (reconstructed images) is an input/output of a noise-reduction super-resolution model serving as the trained model. As illustrated in FIGS. 11 and 12, the data subject to the noise simulation and the resolution simulation is the acquisition data.

The training data generating function 543 is configured to obtain the third projection data. The third projection data corresponds to the second pre-reconstruction data before being reconstructed that corresponds to the noise and the resolution of the second reconstructed image. As illustrated in FIGS. 11 and 12, the third projection data is the projection data having a higher resolution and less noise, being compliant with the second spectral data. As illustrated in FIGS. 11 and 12, the training data generating function 543 is configured to perform a noise simulation on the third projection data, to generate high-resolution high-noise (HR-HN) projection data. Subsequently, the training data generating function 543 is configured to perform a resolution simulation on the HR-HN projection data, to generate the first projection data having a lower resolution and more noise. The first projection data corresponds to the noise and the resolution of the first reconstructed image and corresponds to the first pre-reconstruction data before being reconstructed.

Alternatively, the training data generating function 543 may perform a resolution simulation on the third projection data, to generate low-resolution low-noise (LR-LN) projection data. In that situation, the training data generating function 543 is configured to perform a noise simulation on the LR-LN projection data, to generate the first projection data having a lower resolution and more noise.

In FIG. 11, the model generating function 544 is configured to train the DCNN by using the first projection data and the third projection data to generate a noise-reduction super-resolution model. In that situation, the third projection data corresponds to the first training data, whereas the first projection data corresponds to the second training data. Further, the first projection data corresponds to teacher data in the training of the DCNN. In FIG. 11, the DCNN is trained in a domain of the projection data.

In FIG. 12, the training data generating function 543 is configured to generate a first training image having a higher resolution and less noise, by reconstructing the third projection data. Further, the training data generating function 543 is configured to generate a second training image having a lower resolution and more noise, by reconstructing the first projection data. The first projection data corresponds to the second pre-reconstruction data before being reconstructed that is generated by adding noise to and lowering the resolution of the second pre-reconstruction data. Further, the first pre-reconstruction data before being reconstructed corresponds to the noise and the resolution of the first reconstructed image. The first training image corresponds to the first training data, whereas the second training image corresponds to the second training data. Furthermore, the first training image corresponds to teacher data in the training of the DCNN.

In FIG. 12, the model generating function 544 is configured to train the DCNN by using the first training image and the second training image, to generate a noise-reduction super-resolution model. Unlike in FIG. 11, the DCNN is trained in an image domain in FIG. 12.

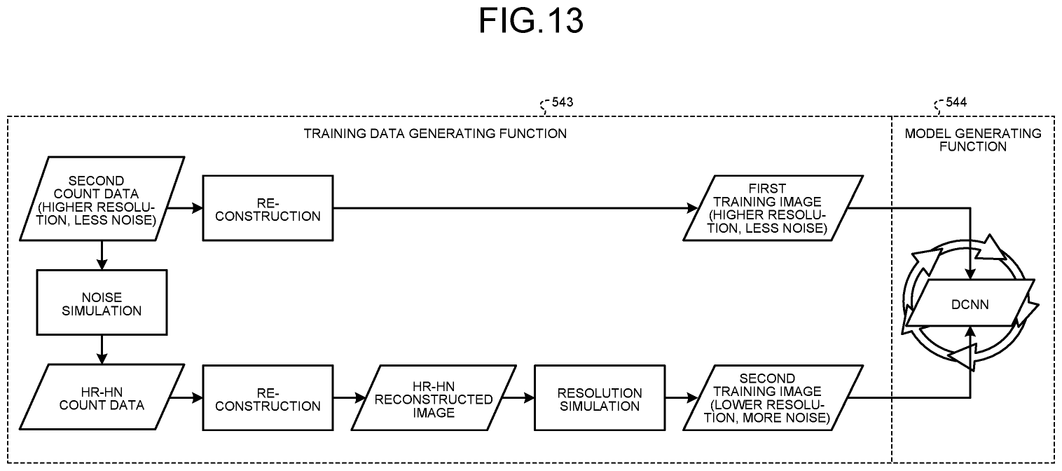

Next, FIG. 10 (b) will be explained, with reference to FIG. 13. FIG. 13 is a diagram illustrating an outline of the model generating process in FIG. 10 (b). As illustrated in FIG. 10 (b) and FIG. 13, the data subject to the noise simulation is the second pre-reconstruction data, whereas the data subject to the resolution simulation is image data. In FIG. 13, it is assumed, as an example, that the second pre-reconstruction data is the second count data.

The training data generating function 543 is configured to obtain the second count data. As illustrated in FIG. 13, the second count data is count data having a higher resolution and less noise, being compliant with the second spectral data. As illustrated in FIG. 13, the training data generating function 543 is configured to generate the first training image by reconstructing the second count data. The training data generating function 543 is configured to perform a noise simulation on the second count data, to generate high-resolution high-noise (HR-HN) count data. Subsequently, the training data generating function 543 is configured to reconstruct the HR-HN count data, to generate an HR-HN reconstructed image. In other words, the training data generating function 543 is configured to generate a noise-added image (the HR-HN reconstructed image) corresponding to the noise of the first reconstructed image, by adding noise to and reconstructing the second pre-reconstruction data (a noise adding process).

The training data generating function 543 is configured to perform a resolution simulation on the HR-HN reconstructed image, to generate the second training image having a lower resolution and more noise. In other words, the training data generating function 543 is configured to generate the second training image corresponding to the noise and the resolution of the first reconstructed image, by lowering the resolution of the noise-added image. Similarly to FIG. 11, the model generating function 544 is configured to train the DCNN by using the first training image and the second training image, to generate a noise-reduction super-resolution model.

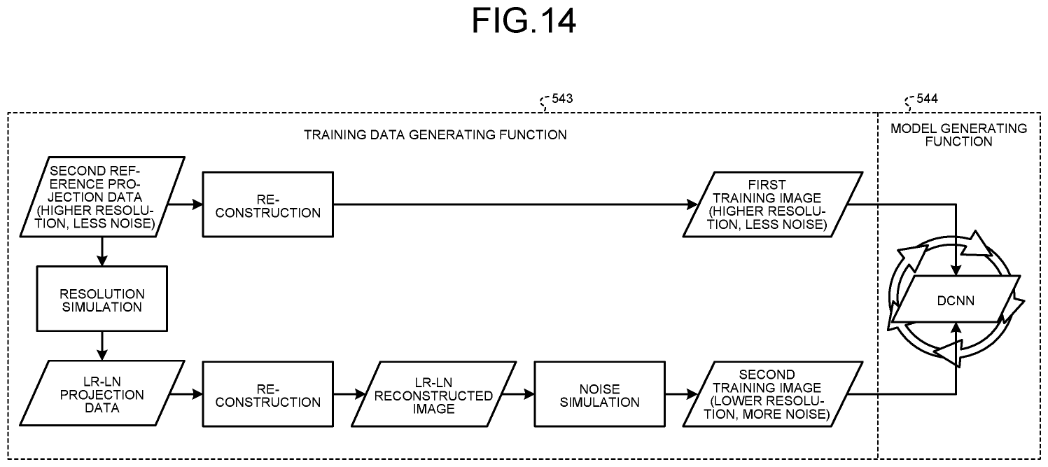

Next, FIG. 10 (c) will be explained with reference to FIG. 14. FIG. 14 is a diagram illustrating an outline of the model generating process in FIG. 10 (c). As illustrated in FIG. 10 (c) and FIG. 14, the data subject to the resolution simulation is the acquisition data, whereas the data subject to the noise simulation is image data. In FIG. 14, the second pre-reconstruction data is, as an example, assumed to be the second reference projection data.

The training data generating function 543 is configured to obtain the second reference projection data. As illustrated in FIG. 14, the second reference projection data is reference projection data having a higher resolution and less noise, being compliant with the second spectral data. As illustrated in FIG. 14, the training data generating function 543 is configured to generate the first training image, by reconstructing the second reference projection data. The first training image is a reference substance image corresponding to the second reference projection data. The training data generating function 543 is configured to perform a resolution simulation on the second reference projection data, to generate low-resolution low-noise (LR-LN) reference projection data. The LR-LN reference projection data corresponds to the first reference projection data. Subsequently, the training data generating function 543 is configured to reconstruct the LR-LN reference projection data, to generate an LR-LN reconstructed image. In other words, the training data generating function 543 is configured to generate the lower resolution image (the LR-LN reconstructed image) corresponding to the resolution of the second reconstructed image, by lowering the resolution and reconstructing the second pre-reconstruction data (i.e., a resolution lowering process).

The training data generating function 543 is configured to perform a noise simulation on the LR-LN reconstructed image, to generate the second training image having a lower resolution and more noise. In other words, the training data generating function 543 is configured to generate the second training image corresponding to the noise and the resolution of the first reconstructed image, by adding noise to the lower resolution image. The second training image illustrated in FIG. 14 corresponds to the reference substance image obtained by reconstructing the first reference projection data. Similarly to FIGS. 12 and 13, the model generating function 544 is configured, as illustrated in FIG. 13, to train the DCNN by using the first training image and the second training image, to generate a noise-reduction super-resolution model.

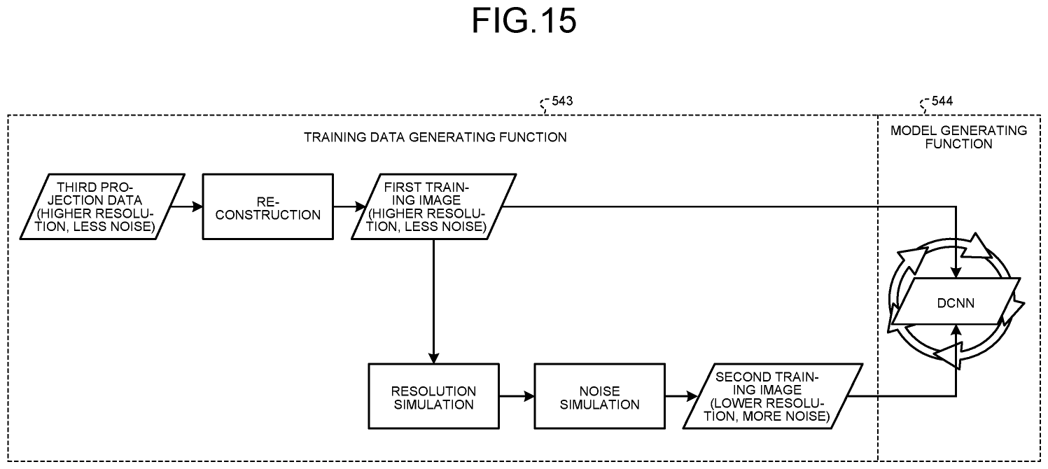

Next, FIG. 10 (d) will be explained, with reference to FIG. 15. FIG. 15 is a diagram illustrating an outline of the model generating process in FIG. 10 (d). As illustrated in FIG. 10 (d) and FIG. 15, the data subject to the noise simulation and the resolution simulation is image data.

The training data generating function 543 is configured to obtain the third projection data. As illustrated in FIG. 15, the third projection data is projection data having a higher resolution and less noise, being compliant with the second spectral data. As illustrated in FIG. 15, the training data generating function 543 is configured to generate the first training image, by reconstructing the third projection data. The training data generating function 543 is configured to sequentially perform a resolution simulation and a noise simulation on the first training image, to generate the second training image having a lower resolution and more noise. In other words, the training data generating function 543 is configured to generate the second training image corresponding to the noise and the resolution of the first reconstructed image, by lowering the resolution of and adding noise to the first training image.

Although FIG. 15 illustrates the procedure in which the resolution simulation is performed, followed by the noise simulation, possible embodiments are not limited to this example. In other words, the training data generating function 543 may be configured to perform the noise simulation on the first training image and to subsequently perform the resolution simulation, so as to generate the second training image. Similarly to FIGS. 12 to 14, the model generating function 544 is configured, as illustrated in FIG. 15, to train the DCNN by using the first training image and the second training image, to generate a noise-reduction super-resolution model.

The model generating method realized by the training apparatus 5 according to the embodiment described above is configured to generate the trained model used in the noise-reduction super-resolution process. For example, the model generating method according to the embodiment includes: generating the second training data corresponding to the noise and the resolution of the first spectral data, by adding the noise to and lowering the resolution of the first training data corresponding to the noise and the resolution of the second spectral data; and generating the trained model to be used in the noise-reduction super-resolution process by training the convolution neural network while using the first training data and the second training data. For example, the model generating method according to the embodiment includes: reconstructing the first training image on the basis of the second pre-reconstruction data before being reconstructed that corresponds to the noise and the resolution of the second reconstructed image; generating the first pre-reconstruction data before being reconstructed that corresponds to the noise and the resolution of the first reconstructed image, by adding the noise to and lowering the resolution of the second pre-reconstruction data; reconstructing the second training image on the basis of the first pre-reconstruction data; and generating the trained model by training the convolution neural network while using the first training image and the second training image.

Further, the model generating method according to the embodiment may include, for example: reconstructing the first training image on the basis of the second pre-reconstruction data before being reconstructed that corresponds to the noise and the resolution of the second reconstructed image; generating the noise-added image corresponding to the noise of the first reconstructed image, by adding the noise to and reconstructing the second pre-reconstruction data; generating the second training image corresponding to the noise and the resolution of the first reconstructed image by lowering the resolution of the noise-added image; and generating the trained model by training the convolution neural network while using the first training image and the second training image. In another example, the model generating method according to the embodiment may include, for example: reconstructing the first training image on the basis of the second pre-reconstruction data before being reconstructed that corresponds to the noise and the resolution of the second reconstructed image; generating the lower resolution image corresponding to the resolution of the first reconstructed image, by lowering the resolution and reconstructing the second pre-reconstruction data; generating the second training image corresponding to the noise and the resolution of the first reconstructed image, by adding the noise to the lower resolution image; and generating the trained model by training the convolution neural network while using the first training image and the second training image.

In yet another example, the model generating method according to the embodiment may include, for example: reconstructing the first training image, on the basis of the second pre-reconstruction data before being reconstructed that corresponds to the noise and the resolution of the second reconstructed image; generating the second training image corresponding to the noise and the resolution of the first reconstructed image, by adding the noise to and lowering the resolution of the first training image; and generating the trained model by training the convolution neural network while using the first training image and the second training image.

As described above, by using the model generating method realized by the training apparatus 5 described herein, it is possible to generate the single trained model (the noise-reduction super-resolution model) capable of realizing, at the same time, both enhancing the spatial resolution (a super resolution) and reducing the noise of the first spectral data, in accordance with the type (e.g., the first pre-reconstruction data (i.e., the first projection data, the second projection data, the first reference projection data, the first count data, etc.) and the first reconstructed image (i.e., the first reference substance image, the first virtual monochrome X-ray image, the first virtual non-contrast-enhanced image, the first iodine map image, the first effective atomic number image, the first electron density image, the first X-ray tube voltage image and the second X-ray tube voltage image, the first energy image, etc.)) of the first spectral data obtained by the spectral medical imaging apparatus. Further, by using the model generating method described herein, it is possible to generate the noise-reduction super-resolution model without being dependent on the type of the training data such as acquisition data or image data. Consequently, by using the model generating method described herein, it is possible to generate the trained model capable of generating the medical image in which visibility is enhanced for objects such as anatomical characteristics in the medical image, while the image quality thereof is also enhanced.

### MODIFICATION EXAMPLES

As a modification example of the present embodiment, the training apparatus 5 may train a DCNN to be a trained model (hereinafter, a super-resolution model) that realizes a super resolution, i.e., increasing a resolution. In that situation, the super-resolution model does not have the function of reducing noise. In this example, the noise simulation in FIGS. 9 to 15 is unnecessary. Further, in the present modification example, the model generating function 544 is configured to carry out the training in an image domain as illustrated in FIGS. 12 to 14. In other words, the super-resolution model is implemented by a medical data processing apparatus in an image domain.

When the super-resolution model in the present modification example is applied, the reconstruction processing function 443 is configured to generate a reconstructed image having a matrix size of 1024×1024, for instance. The data processing function 445 is configured to generate a super-resolution image of the reconstructed image, by inputting the reconstructed image having the matrix size of 1024×1024 to the super-resolution model. In contrast, when the super-resolution model of the present modification example is not applied, the reconstruction processing function 443 is configured to generate a reconstructed image having a matrix size of 512×512. In that situation, the data processing function 445 may generate a noise reduced image of the reconstructed image, by inputting the reconstructed image having the matrix size of 512×512 to a noise reduction model.

### First Application Example

A trained model in the present application example is a model trained by using training data generated by a medical imaging apparatus that uses single energy X-rays. The medical imaging apparatus using the single energy X-rays may be, for example, an X-ray CT apparatus configured to generate single energy X-rays and to perform an imaging process using the generated X-rays on the patient P. A medical data processing apparatus in the present application example is configured to use the second spectral data for visualizing an image related to X-ray spectra from the imaging of the patient P performed by the spectral medical imaging apparatus. In other words, in the present application example, it is possible, similarly to the embodiment, to perform a noise-reduction super-resolution process related to the spectral imaging, by employing the trained model trained while using the training data generated by the medical imaging apparatus that uses the single energy X-rays. Although the trained model in the present application example is not optimal in comparison to the trained model generated in the embodiment, the trained model is effective in reducing the noise and increasing the resolution of the first spectral data, due to the input data. Because the procedure and advantageous effects of the noise-reduction super-resolution process in the present application example are the same as those of the embodiment, explanations thereof will be omitted.

### Second Application Example

A trained model in the present application example is a model trained by using training data generated by a medical imaging apparatus that uses dual energy X-rays. The medical imaging apparatus using the dual energy X-rays is a DECT apparatus. A medical data processing apparatus in the present application example is configured to use the second spectral data for visualizing an image related to X-ray spectra from the imaging of the patient P performed by the spectral medical imaging apparatus. In other words, in the present application example, it is possible, similarly to the embodiment, to perform a noise-reduction super-resolution process related to the spectral imaging, by employing the trained model trained by using the training data generated by the medical imaging apparatus that uses the dual energy X-rays. Although the trained model in the present application example is not optimal in comparison to the trained model generated in the embodiment, the trained model is effective in reducing the noise and increasing the resolution of the first spectral data, due to the input data. Because the procedure and advantageous effects of the noise-reduction super-resolution process in the present application example are the same as those of the embodiment, explanations thereof will be omitted.

### Third Application Example

A trained model in the present application example is a model trained by using training data generated by a Photon Counting X-ray Computed Tomography apparatus (the PCCT apparatus 1). In this situation, the first training data is data acquired from an imaging process performed in a high resolution mode or the like by the PCCT apparatus 1 and is obtained from the PCCT apparatus 1. Further, the second training data may be generated through any of various types of simulations, as explained in the model generating process or may be data acquired from an imaging process performed in a normal resolution mode by the PCCT apparatus 1. A medical data processing apparatus in the present application example is configured to use the second spectral data for visualizing an image related to X-ray spectra from the imaging of the patient P performed by the spectral medical imaging apparatus. In other words, in the present application example, it is possible, similarly to the embodiment, to perform the noise-reduction super-resolution process related to the spectral imaging, by employing the trained model trained by using the training data generated by the PCCT apparatus 1. Although the trained model in the present application example is not optimal in comparison to the trained model generated in the embodiment, the trained model is effective in reducing the noise and increasing the resolution of the first spectral data, due to the input data. Because the procedure and advantageous effects of the noise-reduction super-resolution process in the present application example are the same as those of the embodiment, explanations thereof will be omitted.

### Fourth Application Example

In the present application example, a simulation is performed on image data generated by a DECT apparatus including an Energy Integrated Detector (EID), from scan data (which may be referred to as “count projection data”) obtained by the PCCT apparatus 1. It is assumed that the size (a pixel size) of the X-ray detecting elements in the EID is larger than the size (a pixel size) of the X-ray detecting elements in the X-ray detector 12 included in the PCCT apparatus 1. Further, it is assumed that projection data generated by the EID has more noise than the count projection data does.

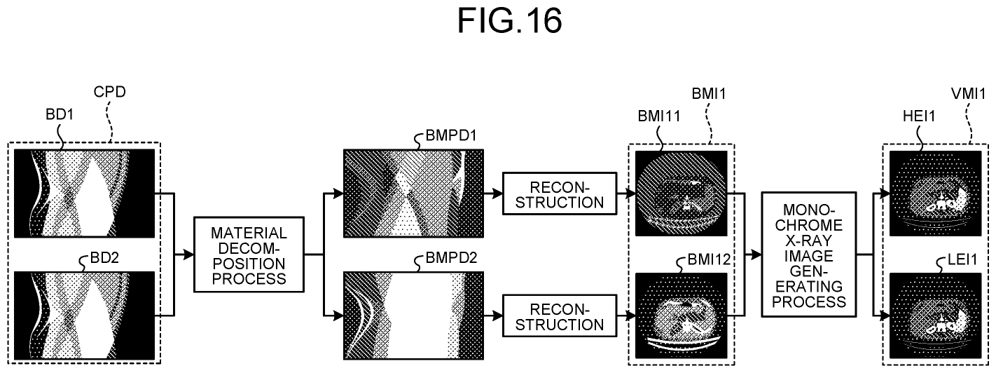

FIG. 16 is a diagram illustrating an example in which a first higher energy monochrome image and a first lower energy monochrome image are generated from count projection data CPD obtained by the PCCT apparatus 1. The first higher energy monochrome image and the first lower energy monochrome image are used for generating trained models corresponding to the energy levels, respectively. As illustrated in FIG. 16, the PCCT apparatus 1 is configured to obtain the count projection data CPD by performing a scan on a patient. In the following sections, to make the explanation simple, it will be assumed that there are two energy bins in the present application example. In other words, by scanning the patient, the PCCT apparatus 1 is configured to obtain first bin data BD1 corresponding to a first energy bin and second bin data BD2 corresponding to the second energy bin, as the count projection data CPD. The first bin data BD1 and the second bin data BD2 each express an X-ray photon count in the corresponding energy bin, together with a view number and an element number.

The data processing function 445 (or the reconstruction processing function 443) is configured to perform a material decomposition process on the first bin data BD1 and the second bin data BD2, to generate raw data BMPD1 of the first reference substance and raw data BMPD2 of the second reference substance. Alternatively, the material decomposition process may be performed by the image processing function 444. Further, because any of known techniques is applicable to the material decomposition process, explanations thereof will be omitted. For example, it is possible to use a method disclosed in Japanese Patent Application Laid-open No. 2020-75078 or a method using a neural network disclosed in the specification of US patent application No. 2015/371378.

In the description above, the example in which the bin data is in the two energy bins was explained; however, the quantity of the energy bins is not limited to two. For instance, the quantity of the energy bins may be five. In that situation, the substance discrimination through the material decomposition is applicable to five reference substances. Preferable quantities of the energy bins are 2 to 5, for example.

In a modification example of the present application example, the data processing function 445 (or the reconstruction processing function 443) may generate first virtual projection data on the basis of the first bin data BD1 and may generate second virtual projection data on the basis of the second bin data BD2. The first virtual projection data is projection data corresponding to virtual first X-ray tube voltage (lower X-ray tube voltage: Low kVp), whereas the second virtual projection data is projection data corresponding to virtual second X-ray tube voltage (higher X-ray tube voltage: High kVp). Further, the first virtual projection data and the second virtual projection data are each projection data having a higher resolution and less noise. In that situation, the data processing function 445 (or the reconstruction processing function 443) is configured to perform a material decomposition process on the first virtual projection data and the second virtual projection data, to generate the raw data BMPD1 of the first reference substance and the raw data BMPD2 of the second reference substance.

As illustrated in FIG. 16, the reconstruction processing function 443 is configured to perform a reconstructing process on the raw data BMPD1 of the first reference substance and on the raw data BMPD2 of the second reference substance, to generate the first reference substance image BMI11 corresponding to the first substance and the second reference substance image BMI12 corresponding to the second substance. The first reference substance image BMI11 corresponding to the first substance and the second reference substance image BMI12 corresponding to the second substance are the first reference substance images BMI1 corresponding to the two types of reference substances.

As illustrated in FIG. 16, the data processing function 445 (or the image processing function 444) is configured to perform a monochrome X-ray image generating process on the first reference substance image BMI11 corresponding to the first substance and the second reference substance image BMI12 corresponding to the second substance, to generate the first virtual monochrome X-ray images VMI1 having mutually-different levels of energy. The first virtual monochrome X-ray images VMI1 include the first higher energy monochrome image HEI1 and the first lower energy monochrome image LEI1. The first virtual monochrome X-ray images VMI1 correspond to the first reconstructed image. Alternatively, the monochrome X-ray image generating process may be performed by the reconstruction processing function 443. Further, because any of known techniques is applicable to the monochrome X-ray image generating process, explanations thereof will be omitted.

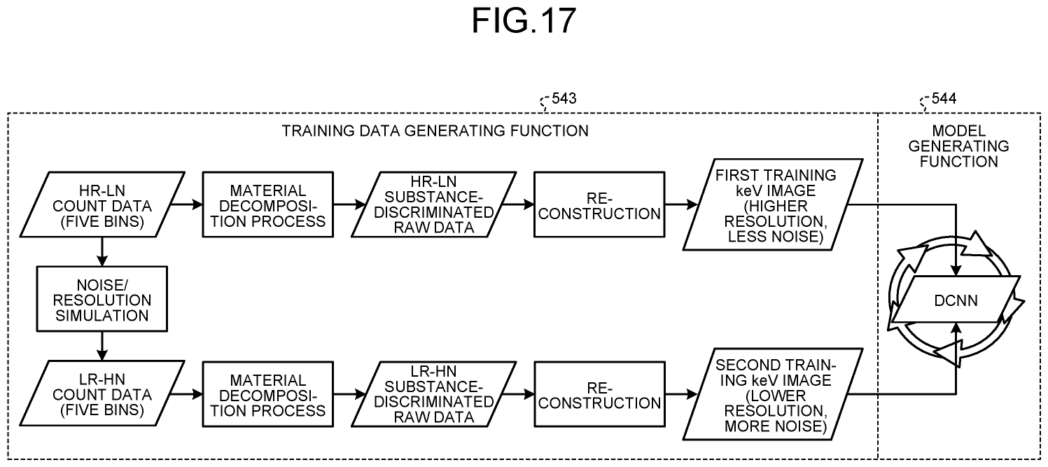

Next, a process of generating the trained model in the present application example will be explained. FIG. 17 is a diagram illustrating an example of an outline of the model generating process. As illustrated in FIG. 17, the data subject to a noise simulation and a resolution simulation (a noise/resolution simulation) may be, for example, High-Resolution Low-Noise (HR-LN) count data (count projection data) CPD from five bins. The HR-LN count data is count data having a higher resolution and less noise and corresponds to the second count data, i.e., the count projection data CPD. The count projection data CPD is the count data having the higher resolution and the less noise, being compliant with the second spectral data. Although FIG. 17 indicates that the HR-LN count data corresponds to the five bins, possible embodiments are not limited to this example. The data may be count projection data corresponding to bins (e.g., two bins) that are not five bins.

As illustrated in FIG. 17, the training data generating function 543 is configured to obtain the HR-LN count data from the PCCT apparatus 1. The training data generating function 543 is configured to perform a material decomposition process on the HR-LN count data, to generate High-Resolution Low-Noise (HR-LN) substance-discriminated raw data. When the HR-LN count data includes five pieces of count projection data belonging to the five bins, the training data generating function 543 is capable of generating five pieces of HR-LN substance-discriminated raw data that discriminate five substances.

As illustrated in FIG. 17, by reconstructing the HR-LN substance-discriminated raw data, the training data generating function 543 is configured to generate a first training keV image corresponding to a prescribed X-ray energy level (keV). The first training keV image corresponds to the first virtual monochrome X-ray image. Further, the training data generating function 543 may generate the first virtual monochrome X-ray image corresponding to another X-ray energy level, by reconstructing the HR-LN substance-discriminated raw data. To this reconstruction, any of known processes is applicable such as an analytical reconstruction based on a Filtered Backprojection (FBP) method or the like, a model-based successive approximation reconstruction, or a deep neural network that receives an input of projection data and outputs a reconstructed image, for example. Further, to any of these reconstructions methods, various types of processes such as a noise reduction process may be applied.

As illustrated in FIG. 17, the training data generating function 543 is configured to perform a noise/resolution simulation on the HR-LN count data. The noise/resolution simulation corresponds to performing the noise simulation (the noise adding process) and the resolution simulation (the resolution lowering process) described above. For example, the training data generating function 543 is configured to perform the noise/resolution simulation on each of the plurality of pieces of bin data of the HR-LN count data. As a result, the training data generating function 543 generates Low-Resolution High-Noise (LR-HN) count data.

The noise/resolution simulation performed on the bin data corresponding to each of the plurality of bins may be the same or may be different among the plurality of bins. For example, the training data generating function 543 is configured to add the noise to the plurality of pieces of data corresponding to the plurality of bins in the HR-LN count data in such a manner that the lower energy the bin corresponds to, the more noise is added to the bin data. In other words, the training data generating function 543 is configured to vary the added noise among the pieces of bin data, in accordance with the energy level related to each of the pieces of bin data (each of the energy bins). As a result, it is possible to cause the data used for the training of the DCNN to be close to the noise characteristics of the image data generated by a DECT apparatus including the EID, for example. In other words, it is possible to cause the data used for the training of the DCNN to reflect the tendency of normal X-ray CT apparatuses and DE apparatuses where the image quality tends to be worse on the lower energy side.

As illustrated in FIG. 17, the training data generating function 543 is configured to perform a material decomposition process on the LR-HN count data, to generate Low-Resolution High-Noise (LR-HN) substance-discriminated raw data. When the LR-HN count data includes five pieces of count projection data belonging to the five bins, the training data generating function 543 is capable of generating five pieces of LR-HN substance-discriminated raw data that discriminate five substances.

As illustrated in FIG. 17, by reconstructing the LR-HN substance-discriminated raw data, the training data generating function 543 is configured to generate second training keV image corresponding to a prescribed X-ray energy level (keV). The second training keV image is a second virtual monochrome X-ray image corresponding to the X-ray energy substantially equal to that of the first virtual monochrome X-ray image. Further, the training data generating function 543 may generate the second virtual monochrome X-ray image corresponding to another X-ray energy level, by reconstructing the LR-HN substance-discriminated raw data. To this reconstruction, any of known processes is applicable such as an analytical reconstruction based on a Filtered Backprojection (FBP) method or the like; a model-based successive approximation reconstruction, or a deep neural network that receives an input of projection data and outputs a reconstructed image, for example. Further, to any of these reconstructions methods, various types of processes such as a noise reduction process may be applied.

Further, although FIG. 17 illustrates the procedure in which the reconstructing process is performed after the material decomposition process, the process of generating the first training keV image and the second training keV image is not limited to this example. For instance, it is also acceptable to perform a reconstructing process on the HR-LN count data so as to generate a plurality of images corresponding to the plurality of bins and to subsequently generate a first training keV image by performing a material decomposition process. Further, it is also acceptable to perform a reconstructing process on the LR-HN count data so as to generate a plurality of images corresponding to the plurality of bins and to subsequently generate a second training keV image by performing a material decomposition process.

Similarly to FIG. 11, the model generating function 544 is configured to train a DCNN by using the first training keV image and the second training keV image, to generate a noise-reduction super-resolution model. In other words, the trained model in the present application example is trained based on the first virtual monochrome X-ray image (the first training keV image) generated on the basis of the count projection data related to the patient P imaged by the photon counting X-ray computed tomography apparatus (the PCCT apparatus); and the second virtual monochrome X-ray image (the second training keV image) obtained by applying, to the count projection data, the simulation process (the noise/resolution simulation process) including the resolution lowering process and the noise adding process performed on the count projection data.

Further, the first virtual monochrome X-ray image (the first training keV image) in the present application example may include a plurality of first virtual monochrome X-ray images corresponding to a plurality of X-ray energy levels, resulting from a material decomposition process performed on a plurality of pieces of bin data corresponding to a plurality of energy bins. Similarly, the second virtual monochrome X-ray image (the second training keV image) in the present application example may also include a plurality of second virtual monochrome X-ray images corresponding to a plurality of X-ray energy levels. In these situations, the trained model in the present application example includes a plurality of trained models corresponding to the plurality of X-ray energy levels. In this situation, the plurality of trained models are trained by using the plurality of first virtual monochrome X-ray images and the plurality of second virtual monochrome X-ray images in correspondence with the plurality of X-ray energy levels, respectively.

Because advantageous effects of the present application example are the same as those of the embodiment and the like, explanations thereof will be omitted.

When technical concept of the embodiment is realized as a medical data processing method, the medical data processing method includes outputting the second spectral data by inputting the first spectral data related to the patient P imaged by the spectral medical imaging apparatus, to the trained model configured to generate, on the basis of the first spectral data, the second spectral data having less noise than the first spectral data and a more super resolution than the first spectral data. The first spectral data corresponds to the medical data obtained from the spectral scan performed on the patient P. The trained model is configured to perform the noise reducing process and the super-resolution process on the first spectral data. Because the procedure and advantageous effects of the noise-reduction super-resolution process implemented by using the medical data processing method are the same as those of the embodiment, explanations thereof will be omitted.

When technical concept of the embodiment is realized as a medical data processing apparatus, the medical data processing apparatus includes a data processing unit configured to output the second spectral data by inputting the first spectral data related to the patient P imaged by the spectral medical imaging apparatus, to the trained model configured to generate, on the basis of the first spectral data, the second spectral data having less noise than the first spectral data and a more super resolution than the first spectral data. The first spectral data corresponds to the medical data obtained from the spectral scan performed on the patient P. The trained model is configured to perform the noise reducing process and the super-resolution process on the first spectral data. Because the procedure and advantageous effects of the noise-reduction super-resolution process performed by the medical data processing apparatus are the same as those of the embodiment, explanations thereof will be omitted.

When technical concept of the present embodiment is realized as a medical data processing program, the medical data processing program causes a computer to realize outputting the second spectral data by inputting the first spectral data related to the patient P imaged by the spectral medical imaging apparatus, to the trained model configured to generate, on the basis of the first spectral data, the second spectral data having less noise than the first spectral data and a more super resolution than the first spectral data. The first spectral data corresponds to the medical data obtained from the spectral scan performed on the patient P. The trained model is configured to perform the noise reducing process and the super-resolution process on the first spectral data. The medical data processing program is, for example, stored in a non-volatile computer-readable storage medium.

For example, it is also possible to realize the noise-reduction super-resolution process by installing the medical data processing program from a non-volatile storage medium into any of various types of server apparatuses (processing apparatuses) related to the medical data processing and further loading the program into the memory. In that situation, the program capable of causing a computer to implement the method may be distributed as being stored in a storage medium such as a magnetic disk (e.g., a hard disk), an optical disc (e.g., a Compact Disc Read-Only Memory (CD-ROM), a DVD, etc.), or a semiconductor memory. Because the processing procedure and advantageous effects of the medical data processing program are the same as those of the embodiment, explanations thereof will be omitted.

According to at least one aspect of the embodiments and the like described above, it is possible to generate the spectral medical image in which visibility is enhanced for objects such as anatomical characteristics in the spectral medical image generated by spectral imaging, while the image quality thereof is also enhanced.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

In relation to the embodiments described above, the following notes are presented as a number of aspects and selected characteristics of the present disclosure:

### Note 1:

A medical data processing method including: outputting second spectral data by inputting first spectral data related to an examined subject imaged by a spectral medical imaging apparatus to a trained model configured to generate, on the basis of the first spectral data, the second spectral data having less noise than the first spectral data and a higher resolution than the first spectral data. The first spectral data corresponds to medical data obtained by performing a spectral scan on the examined subject. The trained model is configured to perform a noise reducing process and a resolution increasing process on the first spectral data.

### Note 2:

The first spectral data may be first pre-reconstruction data before being reconstructed that is acquired from an imaging process performed on the examined subject by the spectral medical imaging apparatus. The second spectral data may be second pre-reconstruction data before being reconstructed. A medical image may be generated on the basis of the second pre-reconstruction data before being reconstructed.

### Note 3:

The first pre-reconstruction data may correspond to first projection data acquired by the spectral medical imaging apparatus at first X-ray tube voltage and to second projection data acquired at second X-ray tube voltage higher than the first X-ray tube voltage. The second pre-reconstruction data may correspond to third projection data corresponding to the first projection data and to fourth projection data corresponding to the second projection data.

### Note 4:

The first pre-reconstruction data may be first reference projection data corresponding to each of a plurality of reference substances. The second pre-reconstruction data may be second reference projection data corresponding to the first reference projection data.

Note 5 The first pre-reconstruction data may be first count data corresponding to each of a plurality of energy ranges. The second pre-reconstruction data may be second count data corresponding to the first count data.

### Note 6:

The first spectral data may be a first reconstructed image reconstructed on the basis of acquisition data acquired from an imaging process performed on the examined subject by the spectral medical imaging apparatus. The second spectral data may be a second reconstructed image having less noise than the first reconstructed image and a higher resolution than the first reconstructed image.

### Note 7:

The first reconstructed image may be represented by a plurality of first reference substance images corresponding to a plurality of reference substances. The second reconstructed image may be represented by a plurality of second reference substance images corresponding to the plurality of first reference substance images.

### Note 8:

The first reconstructed image may be at least one first virtual monochrome X-ray image having a different X-ray energy level. The second reconstructed image may be a second virtual monochrome X-ray image corresponding to the first virtual monochrome X-ray image.

### Note 9:

The first reconstructed image may be a first virtual non-contrast-enhanced image. The second reconstructed image may be a second virtual non-contrast-enhanced image corresponding to the first virtual non-contrast-enhanced image.

### Note 10:

The first reconstructed image may be a first iodine map image. The second reconstructed image may be a second iodine map image corresponding to the first iodine map image.

### Note 11:

The first reconstructed image may be a first effective atomic number image. The second reconstructed image may be a second effective atomic number image corresponding to the first effective atomic number image.

### Note 12:

The first reconstructed image may be a first electron density image. The second reconstructed image may be a second electron density image corresponding to the first electron density image.

### Note 13:

The first reconstructed image may be represented by a plurality of first energy images corresponding to a plurality of energy ranges. The second reconstructed image may be represented by a plurality of second energy images corresponding to the plurality of first energy images.

### Note 14:

The first reconstructed image may be represented by a first X-ray tube voltage image corresponding to first X-ray tube voltage used in an imaging process performed by the spectral medical imaging apparatus and a second X-ray tube voltage image corresponding to second X-ray tube voltage higher than the first X-ray tube voltage. The second reconstructed image may be represented by a third X-ray tube voltage image corresponding to the first X-ray tube voltage image and a fourth X-ray tube voltage image corresponding to the second X-ray tube voltage image.

### Note 15:

The trained model may be a model trained by using training data generated by a medical imaging apparatus that uses single energy X-rays. The second spectral data may be used for visualizing an image related to X-ray spectra from an imaging process performed on the examined subject by the spectral medical imaging apparatus.

### Note 16:

The trained model may be a model trained by using training data generated by a medical imaging apparatus that uses dual energy X-rays. The second spectral data may be used for visualizing an image related to X-ray spectra from an imaging process performed on the examined subject by the spectral medical imaging apparatus.

### Note 17:

The trained model may be a model trained by using training data generated by a photon counting X-ray computed tomography apparatus. The second spectral data may be used for visualizing an image related to X-ray spectra from an imaging process performed on the examined subject by the spectral medical imaging apparatus.

### Note 18:

A model generating method for generating the trained model in the medical data processing method according to any one of Notes 1 to 14, the model generating method including: generating second training data corresponding to noise and a resolution of the first spectral data, by adding noise to and lowering a resolution of first training data corresponding to noise and a resolution of the second spectral data; and generating the trained model by training a convolution neural network while using the first training data and the second training data.

### Note 19:

A model generating method for generating the trained model in the medical data processing method according to any one of Notes 6 to 14, the model generating method including: generating first pre-reconstruction data before being reconstructed that corresponds to noise and a resolution of the first reconstructed image, by adding noise to and lowering a resolution of the second pre-reconstruction data before being reconstructed that corresponds to the noise and the resolution of the second reconstructed image; and generating the trained model by training a convolution neural network while using the first pre-reconstruction data and the second pre-reconstruction data.

### Note 20:

A model generating method for generating the trained model in the medical data processing method according to any one of Notes 6 to 14, the model generating method including: generating, as a first training image, first pre-reconstruction data before being reconstructed that corresponds to noise and a resolution of the first reconstructed image, by adding noise to and lowering a resolution of the second pre-reconstruction data before being reconstructed that corresponds to the noise and the resolution of the second reconstructed image; reconstructing the first training image on the basis of the second pre-reconstruction data; reconstructing the second training image on the basis of the first pre-reconstruction data; and generating the trained model by training a convolution neural network while using the first training image and the second training image.

### Note 21:

A model generating method for generating the trained model in the medical data processing method according to any one of Notes 6 to 14, the model generating method including: reconstructing a first training image on the basis of the second pre-reconstruction data before being reconstructed that corresponds to the noise and the resolution of the second reconstructed image; generating a noise-added image corresponding to noise of the first reconstructed image, by adding noise to and reconstructing the second pre-reconstruction data; generating a second training image corresponding to the noise and a resolution of the first reconstructed image, by lowering a resolution of the noise-added image; and generating the trained model by training a convolution neural network while using the first training image and the second training image.

### Note 22:

A model generating method for generating the trained model in the medical data processing method according to any one of Notes 6 to 14, the model generating method including: reconstructing a first training image on the basis of the second pre-reconstruction data before being reconstructed that corresponds to the noise and the resolution of the second reconstructed image; generating a lower resolution image corresponding to a resolution of the first reconstructed image, by lowering a resolution and reconstructing the second pre-reconstruction data; generating a second training image corresponding to noise and the resolution of the first reconstructed image, by adding noise to the lower resolution image; and generating the trained model by training a convolution neural network while using the first training image and the second training image.

### Note 23:

A model generating method for generating the trained model in the medical data processing method according to any one of Notes 6 to 14, the model generating method including: reconstructing a first training image on the basis of the second pre-reconstruction data before being reconstructed that corresponds to the noise and the resolution of the second reconstructed image; generating a second training image corresponding to noise and a resolution of the first reconstructed image by adding noise to and lowering a resolution of the first training image; and generating the trained model by training a convolution neural network while using the first training image and the second training image.

### Note 24:

A medical data processing apparatus including processing circuitry configured to output second spectral data by inputting first spectral data related to an examined subject imaged by a spectral medical imaging apparatus to a trained model that generates, on the basis of the first spectral data, the second spectral data having less noise than the first spectral data and a higher resolution than the first spectral data. The first spectral data corresponds to medical data obtained by performing a spectral scan on the examined subject. The trained model is configured to perform a noise reducing process and a resolution increasing process on the first spectral data.

### Note 25:

A medical data processing program that causes a computer to realize:
outputting second spectral data by inputting first spectral data related to an examined subject imaged by a spectral medical imaging apparatus to a trained model that generates, on the basis of the first spectral data, the second spectral data having less noise than the first spectral data and a higher resolution than the first spectral data. The first spectral data corresponds to medical data obtained by performing a spectral scan on the examined subject. The trained model is configured to perform a noise reducing process and a resolution increasing process on the first spectral data.

What is claimed is:

1. A medical data processing method comprising:
outputting second spectral data by inputting first spectral data related to an examined subject imaged by a spectral medical imaging apparatus to a trained model configured to generate, on a basis of the first spectral data, the second spectral data having less noise than the first spectral data and a higher resolution than the first spectral data, wherein
the first spectral data corresponds to medical data obtained by performing a spectral scan on the examined subject, and
the trained model is configured to perform a noise reducing process and a super-resolution process on the first spectral data.

2. The medical data processing method according to claim 1, wherein
the first spectral data is first pre-reconstruction data before being reconstructed that is acquired from an imaging process performed on the examined subject by the spectral medical imaging apparatus,
the second spectral data is second pre-reconstruction data before being reconstructed, and
a medical image is generated on a basis of the second pre-reconstruction data before being reconstructed.

3. The medical data processing method according to claim 2, wherein
the first pre-reconstruction data is one selected from among: first projection data acquired by the spectral medical imaging apparatus at first X-ray tube voltage and second projection data acquired at second X-ray tube voltage higher than the first X-ray tube voltage; first reference projection data corresponding to each of a plurality of reference substances; and first count data corresponding to each of a plurality of energy ranges,
the second pre-reconstruction data is one selected from among: third projection data corresponding to the first projection data and fourth projection data corresponding to the second projection data; second reference projection data corresponding to the first reference projection data; and second count data corresponding to the first count data,
when the first projection data and the second projection data are input to the trained model, the third projection data and the fourth projection data are output,
when the first reference projection data is input to the trained model, the second reference projection data is output, and
when the first count data is input to the trained model, the second count data is output.

4. The medical data processing method according to claim 1, wherein
the first spectral data is a first reconstructed image reconstructed on a basis of acquisition data acquired from an imaging process performed on the examined subject by the spectral medical imaging apparatus, and
the second spectral data is a second reconstructed image having less noise than the first reconstructed image and a higher resolution than the first reconstructed image.

5. The medical data processing method according to claim 4, wherein
the first reconstructed image is one selected from among: a plurality of first reference substance images corresponding to a plurality of reference substances; at least one first virtual monochrome X-ray image having a different X-ray energy level; a first virtual non-contrast-enhanced image; a first iodine map image; a first effective atomic number image; a first electron density image; a plurality of first energy images corresponding to a plurality of energy ranges; a first X-ray tube voltage image corresponding to first X-ray tube voltage used in the imaging process performed by the spectral medical imaging apparatus and a second X-ray tube voltage image corresponding to second X-ray tube voltage higher than the first X-ray tube voltage,
the second reconstructed image is one selected from among: a plurality of second reference substance images corresponding to the plurality of first reference substance images; a second virtual monochrome X-ray image corresponding to the first virtual monochrome X-ray image; a second virtual non-contrast-enhanced image corresponding to the first virtual non-contrast-enhanced image; a second iodine map image corresponding to the first iodine map image; a second effective atomic number image corresponding to the first effective atomic number image; a second electron density image corresponding to the first electron density image; a plurality of second energy images corresponding to the plurality of first energy images; a third X-ray tube voltage image corresponding to the first X-ray tube voltage image and a fourth X-ray tube voltage image corresponding to the second X-ray tube voltage image,
when the plurality of first reference substance image are input to the trained model, the plurality of second reference substance images are output,
when the first virtual monochrome X-ray image is input to the trained model, the second virtual monochrome X-ray image is output,
when the first virtual non-contrast-enhanced image is input to the trained model, the second virtual non-contrast-enhanced image is output,
when the first iodine map image is input to the trained model, the second iodine map image is output,
when the first effective atomic number image is input to the trained model, the second effective atomic number image is output,
when the first electron density image is input to the trained model, the second electron density image is output,
when the plurality of first energy images are input to the trained model, the plurality of second energy images is output, and
when the first X-ray tube voltage image and the second X-ray tube voltage image are input to the trained model, the third X-ray tube voltage image and the fourth X-ray tube voltage image are output.

6. The medical data processing method according to claim 4, wherein
the first reconstructed image is represented by a first X-ray tube voltage image corresponding to first X-ray tube voltage used in the imaging process performed by the spectral medical imaging apparatus and a second X-ray tube voltage image corresponding to second X-ray tube voltage higher than the first X-ray tube voltage, and
the second reconstructed image is represented by a third X-ray tube voltage image corresponding to the first X-ray tube voltage image and a fourth X-ray tube voltage image corresponding to the second X-ray tube voltage image.

7. The medical data processing method according to claim 1, wherein
the trained model is a model trained by using training data generated by a medical imaging apparatus that uses single energy X-rays, and
the second spectral data is used for visualizing an image related to X-ray spectra from an imaging process performed on the examined subject by the spectral medical imaging apparatus.

8. The medical data processing method according to claim 1, wherein
the trained model is a model trained by using training data generated by a medical imaging apparatus that uses dual energy X-rays, and
the second spectral data is used for visualizing an image related to X-ray spectra from an imaging process performed on the examined subject by the spectral medical imaging apparatus.

9. The medical data processing method according to claim 1, wherein
the trained model is a model trained by using training data generated by a photon counting X-ray computed tomography apparatus, and
the second spectral data is used for visualizing an image related to X-ray spectra from an imaging process performed on the examined subject by the spectral medical imaging apparatus.

10. The medical data processing method according to claim 1, wherein
the spectral medical imaging apparatus is a dual energy computed tomography apparatus that uses dual energy X-rays, and
the trained model is trained on a basis of:
a first virtual monochrome X-ray image generated on a basis of count projection data related to the examined subject imaged by a photon counting X-ray computed tomography apparatus; and
a second virtual monochrome X-ray image obtained by applying, to the count projection data, a simulation process including a resolution lowering process and a noise adding process performed on the count projection data.

11. The medical data processing method according to claim 10, wherein
the first virtual monochrome X-ray image includes a plurality of first virtual monochrome X-ray images corresponding to a plurality of X-ray energy levels,
the second virtual monochrome X-ray image includes a plurality of second virtual monochrome X-ray images corresponding to the plurality of X-ray energy levels,
the trained model includes a plurality of trained models corresponding to the plurality of X-ray energy levels, and
the plurality of trained models are trained by using the plurality of first virtual monochrome X-ray images and the plurality of second virtual monochrome X-ray images in correspondence with the plurality of X-ray energy levels, respectively.

12. A model generating method for generating a trained model configured, on a basis of first spectral data related to an examined subject imaged by a spectral medical imaging apparatus, to generate the second spectral data having less noise than the first spectral data and a higher resolution than the first spectral data, the model generating method comprising:
generating second training data corresponding to noise and a resolution of the first spectral data, by adding noise to and lowering a resolution of first training data corresponding to the noise and the resolution of the second spectral data; and
generating the trained model by training a convolution neural network while using the first training data and the second training data.

13. The model generating method according to claim 12, wherein
the first spectral data is a first reconstructed image reconstructed on a basis of acquisition data acquired from an imaging process performed on the examined subject by the spectral medical imaging apparatus,
the second spectral data is a second reconstructed image having less noise than the first reconstructed image and a higher resolution than the first reconstructed image, and
the model generating method comprises:
generating first pre-reconstruction data before being reconstructed that corresponds to noise and a resolution of the first reconstructed image, by adding noise to and lowering a resolution of the second pre-reconstruction data before being reconstructed that corresponds to the noise and the resolution of the second reconstructed image;
reconstructing a first training image on a basis of the second pre-reconstruction data;
reconstructing a second training image on a basis of the first pre-reconstruction data; and
generating the trained model by training a convolution neural network while using the first training image and the second training image.

14. A medical data processing apparatus comprising:
processing circuitry configured to output second spectral data by inputting first spectral data related to an examined subject imaged by a spectral medical imaging apparatus to a trained model that generates, on a basis of the first spectral data, the second spectral data having less noise than the first spectral data and a higher resolution than the first spectral data, wherein
the first spectral data corresponds to medical data obtained by performing a spectral scan on the examined subject, and
the trained model is configured to perform a noise reducing process and a super-resolution process on the first spectral data.

15. The medical data processing apparatus according to claim 14, wherein
the first spectral data is first pre-reconstruction data before being reconstructed that is acquired from an imaging process performed on the examined subject by the spectral medical imaging apparatus,
the second spectral data is second pre-reconstruction data before being reconstructed, and
the processing circuitry generates a medical image on a basis of the second pre-reconstruction data before being reconstructed.

16. The medical data processing apparatus according to claim 14, wherein
the first spectral data is a first reconstructed image reconstructed on a basis of acquisition data acquired from an imaging process performed on the examined subject by the spectral medical imaging apparatus, and
the second spectral data is a second reconstructed image having less noise than the first reconstructed image and a higher resolution than the first reconstructed image.

17. The medical data processing apparatus according to claim 14, wherein
the spectral medical imaging apparatus is a dual energy computed tomography apparatus that uses dual energy X-rays, and
the trained model is trained on a basis of:
a first virtual monochrome X-ray image generated on a basis of count projection data related to the examined subject imaged by a photon counting X-ray computed tomography apparatus; and
a second virtual monochrome X-ray image obtained by applying, to the count projection data, a simulation process including a resolution lowering process and a noise adding process performed on the count projection data.

18. The medical data processing apparatus according to claim 17, wherein
the first virtual monochrome X-ray image includes a plurality of first virtual monochrome X-ray images corresponding to a plurality of X-ray energy levels,
the second virtual monochrome X-ray image includes a plurality of second virtual monochrome X-ray images corresponding to the plurality of X-ray energy levels,
the trained model includes a plurality of trained models corresponding to the plurality of X-ray energy levels, and
the plurality of trained models are trained by using the plurality of first virtual monochrome X-ray images and the plurality of second virtual monochrome X-ray images in correspondence with the plurality of X-ray energy levels, respectively.
